(19) United States
(12) Patent Application Publication
(10) Pub. No.: US 2023/0270897 A1
(43) Pub. Date: Aug. 31, 2023

(54) TOXIC SUBJECT DECREASING/ELIMINATING DEVICE

(71) Applicant: NEXT INNOVATION INC., Minato-ku, Tokyo (JP)

(72) Inventor: Hiroshi Michiwaki, Minato-ku, Tokyo (JP)

(21) Appl. No.: 17/999,351

(22) PCT Filed: May 18, 2021
(86) PCT No.: PCT/JP2021/018850

§ 371 Date: Nov. 18, 2022

(30) Foreign Application Priority Data
May 19, 2020 (JP) 2020-087720
Jul. 6, 2020 (JP) 2020-116735
Oct. 5, 2020 (JP) 2020-168628
Nov. 17, 2020 (JP) 2020-190703

(51) Int. Cl. A61L 2/10; A61L 9/20

(52) U.S. Cl. CPC A61L 2/10; A61L 9/20; A61L 2202/11; A61L 2202/15; A61L 2202/14

(57) ABSTRACT

A flow path is disclosed that comprises a suction part that suctions a fluid and a discharge part that discharges the fluid, the flow path being configured so that the path thereof is defined as non-linear and is set so as to be longer than the linear distance of the flow path; and a decreasing/eliminating means in which a subject contained in the fluid and flowing downstream in the flow path is subjected to decomposition and/or deactivation and/or sterilization.

### CROSS-REFERENCE TO RELATED APPLICATIONS

This application is a national phase entry under 35 U.S.C. § 371 of International Patent Application PCT/JP2021/018850, filed May 18, 2021, designating the United States of America and published as International Patent Publication WO 2021/235449 A1 on Nov. 25, 2021, which claims the benefit under Article 8 of the Patent Cooperation Treaty to Japanese Patent Application Serial No. 2020-087720, filed May 19, 2020, Japanese Patent Application Serial No. 2020-116735, filed Jul. 6, 2020, Japanese Patent Application Serial No. 2020-168628, filed Oct. 5, 2020, and Japanese Patent Application Serial No. 2020-190703, filed Nov. 17, 2020.

### TECHNICAL FIELD

The present disclosure relates to a toxic subject decreasing and eliminating device.

### BACKGROUND

In the related art, there is proposed an electric fan that exhibits effects such as dust removal, deodorization, bacteria elimination, antiviral treatment, and antifungal treatment by generating ions (for example, see Patent Document 1). In such an electric fan, an ion generator is embedded in a fan motor and ions are supplied to a fan through an ion outlet provided in a motor housing of the fan motor.

In addition, in an electric fan disclosed in Patent Document 2, an ion generator is provided on a lower side support of a slide pipe, and ions emitted from the ion generator are discharged to the outside using a flow of wind generated by a blower.

Further, in the related art, a fluid sterilization device for sterilizing a fluid flowing a flow path with ultraviolet rays is known. The fluid sterilization device includes a straight pipe and a light source, the light source is disposed on an end portion of the straight pipe to emit ultraviolet light to an inner portion of the straight pipe, thereby performing a sterilization treatment to the fluid such as water flowing through the inside of the straight pipe (for example, see Patent Document 3).

### PRIOR ART DOCUMENT

### Patent Document

Patent Document 1: Japanese Unexamined Patent Publication No. 2008-121579
Patent Document 2: Japanese Unexamined Patent Publication No. 2003-272799
Patent Document 3: Japanese Unexamined Patent Publication No. 2017-064610

### BRIEF SUMMARY

### Technical Goals

The electric fans disclosed in Patent Documents 1 and 2 emit ions to outside using the flow of the wind, however, the amount of generated ions decreases with time. Accordingly, although the flow of the wind is used, it is difficult to fill a space such as a room with ions. Therefore, even if a toxic subject such as germs and viruses that harm the human body exists in the space, it is difficult to obtain an effect of reliably extinguishing, inactivating, decreasing, or eliminating the toxic subject by the ions. Therefore, an air flow is generated while the toxic subject remains in the space, and the blowing of the air by the electric fan in this state scatters, agitates, and diffuses the toxic subject such as droplets, particularly, so-called microdroplets, that are considered to remain for a significantly long time in the space, or viruses attached to an aerosol, in the space. Thus, there is a problem regarding the spread of infection of diseases or the like.

In addition, the fluid sterilization device disclosed in Patent Document 3 emits a predetermined or more amount of ultraviolet rays to a fluid for the sterilization with the ultraviolet rays, and thus, there is a problem that it is quite difficult to continuously emit the ultraviolet rays to the fluid until the sterilization is completed, depending on a length or size of the straight pipe.

The disclosure is made in view of the aforementioned problems through intensive studies of the inventors, and an object thereof is to provide a means for gradually and reliably decreasing and eliminating a toxic subject by sucking a fluid while reliably decomposing, inactivating, and/or causing extinction to decrease and eliminate the toxic subject or the like contained in the fluid and discharging the fluid, in which the toxic subject is decreased or eliminated, to the outside with a simple structure.

### Technical Solutions

A toxic subject decreasing and eliminating device of the present disclosure includes a flow path that communicates a suction portion configured to suck a fluid and a discharge portion configured to discharge the fluid with each other, and defines a path in a non-linear form to be longer than a straight line distance, and a decreasing and eliminating means for decomposing and/or inactivating and/or sterilizing a subject contained in the fluid flowing down in the flow path.

The toxic subject decreasing and eliminating device of the present disclosure may further include a flow generating means for generating a flow of the fluid along the flow path from a side of the suction portion toward a side of the discharge portion.

In the toxic subject decreasing and eliminating device of the present disclosure, the flow generating means may have at least one or more fan structures selected from an axial-flow fan, a centrifugal fan, a mixed flow fan, a centrifugal axial-flow fan, a vortex-flow fan, or a tangential fan.

In the toxic subject decreasing and eliminating device of the present disclosure, the flow generating means may be disposed in the vicinity of the suction portion and/or the discharge portion.

In the toxic subject decreasing and eliminating device of the present disclosure, the flow generating means may have one or more fan structures, and the fan structure may be fixed to a single rotating shaft.

In the toxic subject decreasing and eliminating device of the present disclosure, the flow generating means may include one or more fan structures, a rotating shaft, and a driving motor.

The toxic subject decreasing and eliminating device of the present disclosure may further include at least one sensor of a temperature sensor, a humidity sensor, a human sensor, or a pollution sensor, and a flow generated by the flow generating means may be controlled based on detection by the sensor.

In the toxic subject decreasing and eliminating device of the present disclosure, the flow path may reciprocate in a predetermined direction.

In the toxic subject decreasing and eliminating device of the present disclosure, the predetermined direction may be a horizontal direction and/or a vertical direction.

In the toxic subject decreasing and eliminating device of the present disclosure, the predetermined direction may be a direction parallel to ultraviolet rays emitted by the decreasing and eliminating means.

In the toxic subject decreasing and eliminating device of the present disclosure, the flow path may include a portion extending in a curved shape and/or meander shape.

In the toxic subject decreasing and eliminating device of the present disclosure, the portion extending in the curved shape may form a spiral shape or a vortex shape.

In the toxic subject decreasing and eliminating device of the present disclosure, the flow path may have a distance that is an integer multiple or more of a straight line distance between the suction portion and the discharge portion.

In the toxic subject decreasing and eliminating device of the present disclosure, the flow path may be configured with a flow path defining means.

In the toxic subject decreasing and eliminating device of the present disclosure, the flow path defining means may be configured to include any one or more of a portion of the decreasing and eliminating means, a portion of an inner surface of a housing, and a guide plate.

In the toxic subject decreasing and eliminating device of the present disclosure, the decreasing and eliminating means may include an ultraviolet lamp or an ultraviolet light-emitting diode (LED).

In the toxic subject decreasing and eliminating device of the present disclosure, the ultraviolet lamp may be formed as a cylindrical tube.

In the toxic subject decreasing and eliminating device of the present disclosure, the decreasing and eliminating means may include a reflective surface that reflects ultraviolet rays from the ultraviolet lamp toward the flow path, the reflective surface may have a concave cross section having an elliptical arc shape, and the ultraviolet lamp may be disposed at a focal point position of an ellipse that forms the elliptical arc of the reflective surface.

In the toxic subject decreasing and eliminating device of the present disclosure, a plurality of the ultraviolet LEDs may be arranged approximately in a straight line or arranged to be aligned vertically and/or horizontally in a plane.

In the toxic subject decreasing and eliminating device of the present disclosure, the decreasing and eliminating means may be integrally configured with the flow path.

In the toxic subject decreasing and eliminating device of the present disclosure, the discharge portion may be configured to discharge the fluid toward a suction region by the suction portion.

In the toxic subject decreasing and eliminating device of the present disclosure, the discharge portion may be configured to discharge the fluid toward a region different from a suction region by the suction portion.

In the toxic subject decreasing and eliminating device of the present disclosure, a flow velocity of a fluid discharged by the discharge portion may be slower than a flow velocity of a fluid sucked by the suction portion.

In the toxic subject decreasing and eliminating device of the present disclosure, an area of a discharge port of the fluid in the discharge portion may be larger than an area of a suction port of the fluid in the suction portion.

The toxic subject decreasing and eliminating device of the present disclosure may further include a housing having an approximately tubular shape and elongated shape, one of the suction portion and the discharge portion may be disposed on one end side with respect to a longitudinal central portion of the housing, and the other one may be disposed on the other end portion with respect to the longitudinal central portion of the housing.

In the toxic subject decreasing and eliminating device of the present disclosure, the suction portion may perform high-speed suction, and the discharge portion may perform low-speed discharge.

In the toxic subject decreasing and eliminating device of the present disclosure, the suction portion may perform low-speed suction, and the discharge portion may perform high-speed discharge.

In the toxic subject decreasing and eliminating device of the present disclosure, the suction portion may include a suction port for sucking the fluid from a wide area.

In the toxic subject decreasing and eliminating device of the present disclosure, the suction portion may include a suction port for sucking the fluid from a single direction.

In the toxic subject decreasing and eliminating device of the present disclosure, the suction portion may allow the sucked fluid to flow down to the flow path as a jet flow.

In the toxic subject decreasing and eliminating device of the present disclosure, the discharge portion may include a discharge port for discharging the fluid to a wide area.

In the toxic subject decreasing and eliminating device of the present disclosure, the discharge portion may include a discharge port for discharging the fluid in a single direction.

In the toxic subject decreasing and eliminating device of the present disclosure, the discharge portion may include a continuous or intermittent exhaust port extending in one direction, and an air curtain may be generated by exhaust from the exhaust port.

In the toxic subject decreasing and eliminating device of the present disclosure, the discharge portion may be configured to discharge a jet flow.

In the toxic subject decreasing and eliminating device of the present disclosure, the flow path may be formed of an ultraviolet transmitting material or an ultraviolet reflective material.

In the toxic subject decreasing and eliminating device of the present disclosure, an ultraviolet reflecting means may be disposed on a portion facing the decreasing and eliminating means with the flow path interposed therebetween, and the ultraviolet reflecting means may be configured to reflect ultraviolet rays emitted from the decreasing and eliminating means and transmitted through the flow path toward the flow path.

The toxic subject decreasing and eliminating device of the present disclosure may further include a second decreasing and eliminating means for decreasing and eliminating a subject, and the second decreasing and eliminating means may include an electric field generating means for generating an electric field in a flow path, a heating means for heating inside of a flow path, and/or an ion generating means for generating ions.

In the toxic subject decreasing and eliminating device of the present disclosure, a filter for collecting foreign matters contained in the fluid may be provided in the flow path.

In the toxic subject decreasing and eliminating device of the present disclosure, the flow path may include a cyclone portion for separating foreign matters contained in the fluid from the flow path.

The toxic subject decreasing and eliminating device of the present disclosure may further include a partition configured to partition a space around the device.

The toxic subject decreasing and eliminating device may be embedded in separate equipment.

In the toxic subject decreasing and eliminating device of the present disclosure, the separate equipment may be a roof, seat backrest, seat headrest, concrete panel, air conditioner, table, desk, chair, elevator, plant, septic tank, or pipe.

In the toxic subject decreasing and eliminating device of the present disclosure, a plurality of guide plates for partitioning the flow path into a plurality of regions concentrically may be disposed in the flow path at intervals in a flow direction of the fluid, the guide plate may have a communication path on one end portion or the other end portion along a reciprocating direction of the fluid, and the fluid in the flow path may be allowed to flow down from the suction portion toward the discharge portion while flowing in the reciprocating direction and flowing to an inner side in a radial direction through the communication path.

In the toxic subject decreasing and eliminating device of the present disclosure, each of the regions partitioned by the guide plates may have the same cross-sectional area.

In the toxic subject decreasing and eliminating device of the present disclosure, each of the regions partitioned by the guide plates may have a cross-sectional area set to be narrower toward a downstream side.

In the toxic subject decreasing and eliminating device of the present disclosure, each of the regions partitioned by the guide plates may have a cross-sectional area set to be wider toward a downstream side.

In the toxic subject decreasing and eliminating device of the present disclosure, the suction portion and the discharge portion may be disposed in plurality intermittently along a circumferential direction, the flow path may be partitioned into a plurality of sections in the circumferential direction and may communicate the suction portion and the discharge portion directed in the same direction with each other, and the fluid sucked from one side by the suction portion may be discharged toward the one side through the discharge portion.

In the toxic subject decreasing and eliminating device of the present disclosure, the decreasing and eliminating means may be disposed on a portion surrounded by the flow path.

In the toxic subject decreasing and eliminating device of the present disclosure, the fluid may be a gas, liquid, and/or powder.

In the toxic subject decreasing and eliminating device of the present disclosure, the toxic subject may be bacteria, viruses and/or harmful molecules.

A toxic subject decreasing and eliminating device includes a flow path that communicates a suction portion configured to suck a fluid and a discharge portion configured to discharge the fluid with each other, and is defined in a spiral shape or vortex shape, a reflective layer that is disposed in the flow path and extends along a direction in which the fluid flows, and a decreasing and eliminating means that is disposed on the suction portion and/or the discharge portion and decomposes and/or inactivates and/or sterilizes a subject contained in the fluid flowing down in the flow path with ultraviolet rays, and ultraviolet rays emitted from the decreasing and eliminating means may be reflected by the reflective layer and illuminate approximately an entire region of the flow path.

In the toxic subject decreasing and eliminating device of the present disclosure, the reflective layer may be continuously or intermittently formed in the flow path.

In the toxic subject decreasing and eliminating device of the present disclosure, the flow path having the spiral shape may be formed by stacking partial spiral flow paths each having a spiral shape for one turn in an axial direction.

In the toxic subject decreasing and eliminating device of the present disclosure, the partial spiral flow path may include an insertion portion that is formed in a central portion and through which the decreasing and eliminating means is inserted, a support that fits into another communicating partial spiral flow path and extends in the axial direction, and a recess into which the support provided in the other partial spiral flow path fits.

A toxic subject decreasing and eliminating device includes a flow path that communicates a suction portion configured to suck a fluid and a discharge portion configured to discharge the fluid with each other, a reflective layer that is disposed in the flow path and extends along a direction in which the fluid flows, and a decreasing and eliminating means that is disposed on the suction portion and/or the discharge portion and decomposes and/or inactivates and/or sterilizes a subject contained in the fluid flowing down in the flow path with ultraviolet rays, ultraviolet rays emitted from the decreasing and eliminating means may be reflected by the reflective layer and illuminate approximately an entire region of the flow path.

In the toxic subject decreasing and eliminating device of the present disclosure, an ultraviolet leakage suppressor for suppressing ultraviolet light leaking to outside of the device and transmitting the fluid may be disposed on a side of the suction portion and/or a side of the discharge portion.

In the toxic subject decreasing and eliminating device of the present disclosure, the ultraviolet leakage suppressor may include a plurality of holes forming a honeycomb structure.

In the toxic subject decreasing and eliminating device of the present disclosure, the ultraviolet leakage suppressor may include a light shielding surface having a bent cross section.

In the toxic subject decreasing and eliminating device of the present disclosure, the ultraviolet leakage suppressor may include a first inclined surface and a second inclined surface, and the first inclined surface and the second inclined surface may be disposed at different inclination angles.

In the toxic subject decreasing and eliminating device of the present disclosure, the ultraviolet leakage suppressor may include a first inclined surface and a second inclined surface, and the first inclined surface and the second inclined surface may be spaced apart from each other and provided to be shifted so that one inclined surface exists on an extension line of the other inclined surface in an inclination direction.

### Effects

According to the present disclosure, it is possible to suck a fluid while reliably decomposing, inactivating, and/or causing extinction of the toxic subject or the like contained in the fluid and discharging the fluid, in which the toxic subject is decreased or eliminated, to a space where the toxic subject is less likely to exist, with a simple structure, thereby gradually and reliably decreasing and eliminating the toxic subject without spreading the toxic subject in the space.

### BRIEF DESCRIPTION OF THE DRAWINGS

FIG. 1 is a diagram illustrating a schematic configuration of a toxic subject decreasing and eliminating device of the present disclosure.

FIG. 2 is a diagram illustrating an example of the toxic subject decreasing and eliminating device of the present disclosure.

FIGS. 3A-3C are diagrams illustrating an example of a configuration of a flow path.

FIGS. 4A and 4B are diagrams illustrating another example of the configuration of the flow path.

FIGS. 5A-5D are diagrams illustrating an example of arrangement of a suction portion.

FIGS. 6A-6D are diagrams illustrating an example of arrangement of a discharge portion.

FIG. 7 is a diagram illustrating an example of arrangement of an ultraviolet light source.

FIGS. 8A-8D are diagrams illustrating another example of the arrangement of the ultraviolet light source.

FIG. 9 is a diagram illustrating an example of arrangement of a concave reflector for the ultraviolet light source.

FIG. 10 is a diagram illustrating a reflection direction of ultraviolet rays from the concave reflector.

FIG. 11 is a diagram illustrating a toxic subject decreasing and eliminating device having another configuration.

FIG. 12 is a diagram illustrating a flow direction of a fluid in a flow path and a direction of ultraviolet rays.

FIGS. 13A-13E are diagrams illustrating an example of arrangement of a flow generator.

FIG. 14 is a diagram illustrating an exterior of the toxic subject decreasing and eliminating device having the other configuration.

FIG. 15 is a cross-sectional view illustrating the toxic subject decreasing and eliminating device having the other configuration.

FIG. 16 is a front view illustrating a housing.

FIG. 17A is a plan view of an outer layer portion of a flow path portion and FIG. 17B is a front view of the outer layer portion of the flow path portion.

FIG. 18 is a cross-sectional view illustrating the inside of the flow path portion.

FIG. 19A is a perspective view illustrating a blower, FIG. 19B is a side view illustrating the blower, and FIG. 19C is a cross-sectional view illustrating the blower.

FIG. 20 is a diagram illustrating a flow direction of a fluid in an air flow path.

FIG. 21A is a diagram illustrating an exterior of the toxic subject decreasing and eliminating device and FIG. 21B is a cross-sectional view taken along line A-A of FIG. 21A.

FIG. 22 is a cross-sectional view illustrating the toxic subject decreasing and eliminating device.

FIG. 23 is a diagram illustrating a blower 70.

FIG. 24 is a diagram illustrating another example of the ultraviolet light source.

FIG. 25 is a cross-sectional view illustrating the toxic subject decreasing and eliminating device having another configuration.

FIG. 26 is a diagram illustrating a schematic configuration of a toxic subject decreasing and eliminating device including a cyclone chamber.

FIGS. 27A-27D are diagrams illustrating other configurations of the blower.

FIG. 28 is a diagram illustrating a vortex-shaped flow path.

FIG. 29 is a diagram illustrating a vortex-shaped flow path.

FIG. 30 is a diagram illustrating another vortex-shaped flow path.

FIG. 31 is a perspective view illustrating a partial spiral flow path.

FIG. 32 is a diagram illustrating positional alignment of partial spiral flow paths.

FIG. 33 is a diagram illustrating two connected partial spiral flow paths.

FIG. 34 is a plan view illustrating an ultraviolet leakage suppressor.

FIG. 35 is a perspective view illustrating a toxic subject decreasing and eliminating device that is combined with the ultraviolet leakage suppressor.

FIG. 36A is a plan view illustrating another example of the ultraviolet leakage suppressor and FIG. 36B is a cross-sectional view taken along line A-A.

FIG. 37A is a plan view illustrating another example of the ultraviolet leakage suppressor and FIG. 37B is a cross-sectional view taken along a line A-A.

FIG. 38 is a diagram illustrating another example of the ultraviolet leakage suppressor.

### DETAILED DESCRIPTION

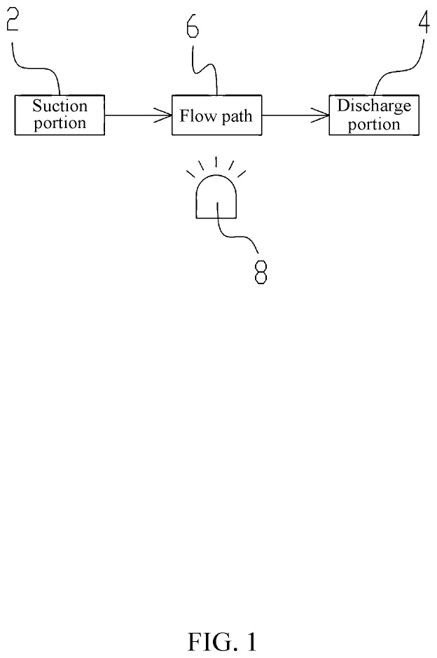

Hereinafter, embodiments of a toxic subject decreasing and eliminating device of the present disclosure will be described with reference to the drawings. FIG. 1 is a diagram illustrating a schematic configuration of a toxic subject decreasing and eliminating device 1 of the present disclosure. The toxic subject decreasing and eliminating device 1 may include a suction portion 2 that sucks a fluid, a discharge portion 4 that discharges the fluid, a flow path 6 that defines a path between the suction portion 2 and the discharge portion 4 in a non-linear shape to set to be longer than a straight line distance to communicate the suction portion 2 and the discharge portion 4 with each other, and an ultraviolet light source 8 (a decreasing and eliminating means) that decreases and eliminates (e.g., decomposes, inactivates, and sterilizes) a toxic subject by irradiating the toxic subject contained in the fluid flowing down in the flow path 6 with ultraviolet rays.

The fluid is a term including a gas, liquid, and powder, and the toxic subject includes not only pathogenic microorganisms such as bacteria and viruses, but also formaldehyde, sulfurous acid gas, nitrous acid gas, or the like containing harmful molecules and is a subject that is at least toxic to the human body and moves along with the fluid.

The suction portion 2 is an opening or nozzle for taking a fluid outside the device into the inside of the device, and the discharge portion 4 is an opening or nozzle for discharging the fluid inside the device to the outside. The toxic subject decreasing and eliminating device 1 performs emission of the ultraviolet rays by the ultraviolet light source 8 while making the fluid taken from the suction portion 2 flow along the flow path 6.

Accordingly, the toxic subject in the fluid may be decreased and eliminated by the ultraviolet rays, and thus, it is possible to prevent the toxic subject from adversely affecting on the human body through the fluid discharged from the discharge portion 4.

In addition, the toxic subject decreasing and eliminating device 1, a flow generating means such as a fan (not shown) may be disposed inside the eliminating device 1 and/or outside the eliminating device 1 in order to take the fluid into the device via the suction portion 2.

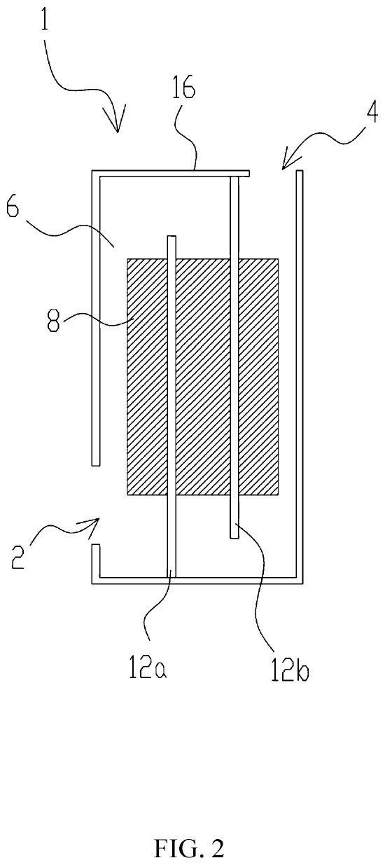

FIG. 2 is a diagram illustrating an example of the toxic subject decreasing and eliminating device 1 of the present disclosure. The toxic subject decreasing and eliminating device 1 includes a housing 16 and the flow path 6 or the ultraviolet light source 8 is disposed in the housing 16. The housing 16 includes the suction portion 2 and the discharge portion 4 spaced apart from each other in an up and down direction. That is, the suction portion 2 is formed by opening a lower portion of an outer periphery of the housing 16, and the discharge portion 4 is formed by opening an upper end surface thereof. In addition, the housing 16 and the flow path 6 may be integrated or separate. The housing 16 is desirably configured to surround at least the flow path 6 and accommodate the ultraviolet light source 8, but a shape thereof is not particularly limited and may be a cylindrical shape, a columnar shape, a rectangular parallelepiped shape, or the like.

In the flow path 6, guide plates 12a and 12b are arranged so that the fluid is able to flow in a reciprocating manner in the up and down direction. That is, the guide plate 12a, of which an upper end portion is spaced apart from a ceiling in the housing 16, and the guide plate 12b, of which a lower end portion is spaced apart from a bottom portion in the housing 16, are alternately disposed so that the fluid reciprocates in the up and down direction.

The flow path 6 is configured with a flow path defining means including some portions of the guide plates 12a and 12b, an inner surface of the housing 16, and the ultraviolet light source 8.

The ultraviolet light source 8 is a light source for emitting the ultraviolet rays such as, for example, a sterilization lamp, an ultraviolet lamp, an ultraviolet light-emitting diode (LED), or the like, and ultraviolet light source 8 emits ultraviolet rays in a wide range within the flow path 6. For example, the ultraviolet light source 8 may be disposed to cross the guide plates 12a and 12b.

The ultraviolet light source 8 performs the decreasing and eliminating of a target toxic subject, such as, decomposition, inactivation, disinfection, sterilization, and the like. Such an ultraviolet ray is desirable to have a wavelength of 250 to 300 nanometers (nm) and more desirable to have a wavelength of approximately 250 to 270 nm. The ultraviolet ray may be a near ultraviolet ray (UV-C) having a wavelength of less than 260 nm, a far ultraviolet ray (a wavelength of 10 to 200 nm), an extreme ultraviolet ray (a wavelength of 10 to 121 nm), or the like, as long as it may at least decrease and eliminate the toxic subject. In addition, the ultraviolet ray may be a near ultraviolet ray (UV-A or UV-B) having a wavelength exceeding 300 nm.

Also, an ultraviolet LED may be applied to the ultraviolet light source 8. As the ultraviolet LED, for example, those using aluminum gallium nitride (AlGaN) may be used. A plurality of ultraviolet LEDs may be, for example, arranged substantially linearly or arranged and aligned vertically and/or horizontally in a plane to form the ultraviolet light source.

According to this configuration, fluids respectively sucked from the suction portion 2 in a plurality of directions may be retained for a sufficient time to decrease and eliminate the toxic subject so as to be continuously irradiated with the ultraviolet rays from the ultraviolet light source 8, compared to a case where the suction portion 2 and the discharge portion 4 are directly connected by a straight line distance. In this case, since the fluid flows in a reciprocating manner along the flow path 6, an exposure time of the toxic subject to the ultraviolet rays may be extended. Also, a direction (posture) of the toxic subject with respect to the ultraviolet light source 8 may change along the flow of the fluid due to the reciprocating flow, and accordingly, the toxic subject is exposed to the ultraviolet rays in various directions. That is, even the toxic subject existing behind dust or dirt may be irradiated with the ultraviolet rays, and therefore, the toxic subject may be reliably decreased and eliminated.

### Description of Flow Path 6

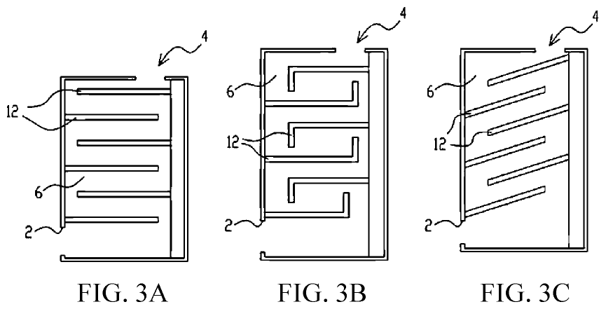

The flow path 6 having the configuration, in which the fluid flows in the up and down direction, has been described but the flow path 6 may have another configuration. For example, the air (fluid) may reciprocate in the flow path 6 in a horizontal direction by arranging a plurality of guide plates 12 shown in FIG. 3A horizontally. As shown in FIG. 3B, the air may flow down in the flow path while reciprocating in the horizontal and up and down directions by arranging the plurality of guide plates 12 each having a substantially L-shaped cross section. As shown in FIG. 3C, the air may flow down in the flow path while reciprocating in a direction inclined from the horizontal direction by arranging the guide plates 12 in parallel in the inclined direction.

In addition, the flow path 6 may be formed to have a portion extending in a curved shape or meander shape or may be formed to set a portion extending in a curved shape to have a spiral shape or a vortex shape, as long as it may set the path communicating the suction portion 2 and the discharge portion 4 to be longer than the straight line distance. Also, a length of the flow path 6 may be, for example, an integer multiple or more of the straight line distance between the suction portion 2 and the discharge portion 4. Further, when the suction portion 2 and the discharge portion 4 are spaced apart from each other in the up and down direction and the flow path reciprocates in the up and down direction, the length of the flow path 6 may be set as approximately an odd-number multiple or the like of the straight line distance between the suction portion 2 and the discharge portion 4. In addition, when the suction portion 2 and the discharge portion 4 are disposed to be close to each other in the up and down direction and the flow path reciprocates in the up and down direction, the length of the flow path 6 may be set to be approximately an even-number multiple or the like of the straight line distance between the suction portion 2 and the discharge portion 4.

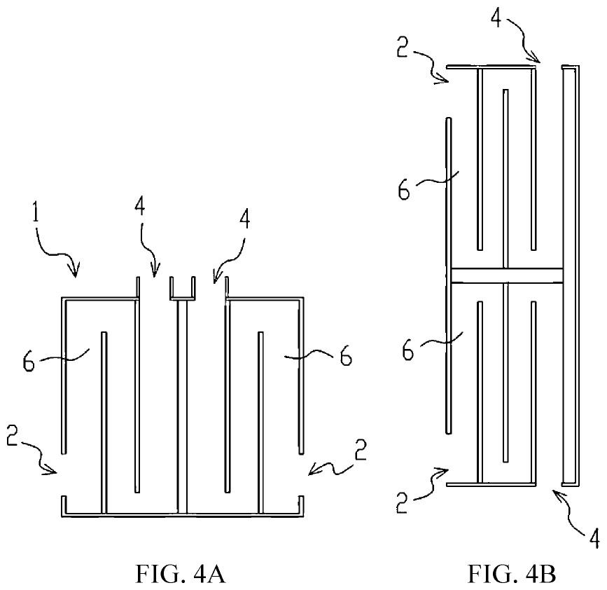

In addition, the number of flow paths 6 disposed in the toxic subject decreasing and eliminating device 1 may be set appropriately. For example, as shown in FIG. 4A, the pair of flow paths 6 may be disposed to face each other with the ultraviolet light source 8 interposed therebetween, or as shown in FIG. 4B, the plurality of flow paths 6 may be disposed to be aligned in the up and down direction. Also, the plurality of flow paths 6 may be disposed in a circumferential direction with respect to the ultraviolet light source 8. As described above, when the plurality of flow paths 6 are provided, the suction portion 2 and the discharge portion 4 may be provided in each flow path 6. The suction portions 2 (or the discharge portions 4) may face the same direction or may face different directions.

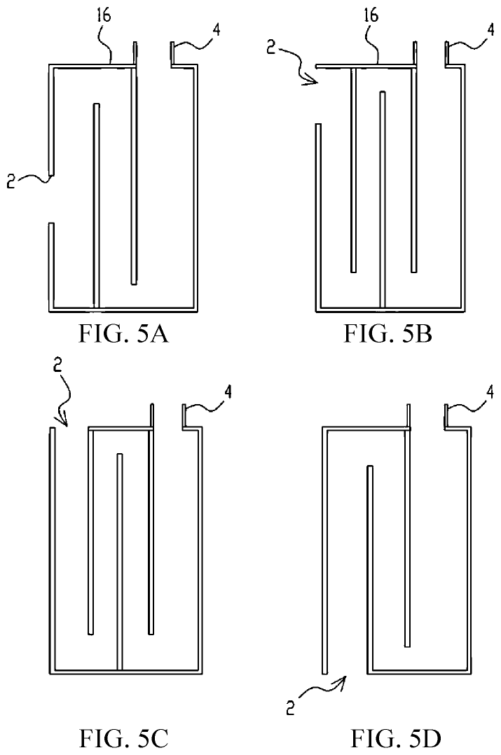

In addition, although it is described that the position of the suction portion 2 is the lower portion of the outer periphery of the housing 16, the position of the suction portion 2 may be set appropriately. For example, as shown in FIG. 5A, the suction portion 2 may be disposed in the middle of the outer periphery of the housing 16, or as shown in FIG. 5B, the suction portion 2 may be disposed on an upper end portion. Also, as shown in FIG. 5C, the suction portion 2 may be disposed on an upper end surface, or as shown in FIG. 5D, the suction portion 2 may be disposed on a lower end surface.

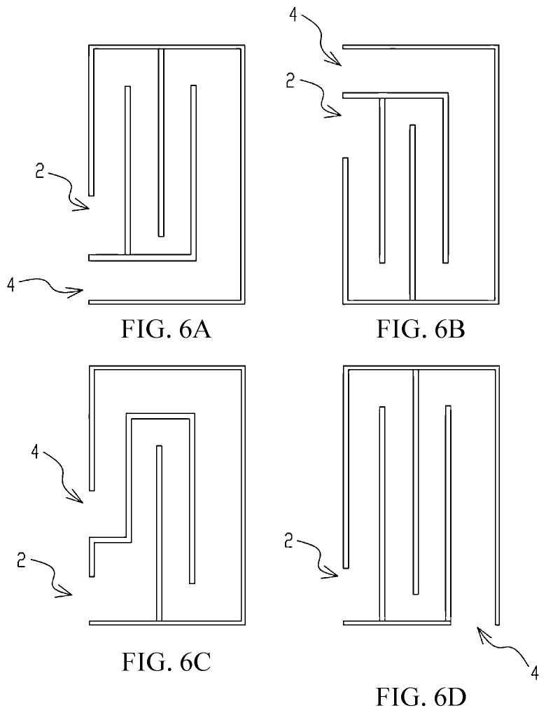

In addition, although it is described that the position of the discharge portion 4 is the upper end surface of the housing 16, the position of the discharge portion 4 may be set appropriately. For example, as shown in FIG. 6A, the discharge portion 4 may be disposed below the suction portion 2 as the lower portion of the outer periphery of the housing 16, and as shown in FIG. 6B, the discharge portion 4 may be disposed on the upper end portion. In addition, as shown in FIG. 6C, the discharge portion 4 may be disposed in the middle of the outer periphery of the housing 16, and as shown in FIG. 6D, the discharge portion 4 may be disposed on the lower end surface of the housing 16. Accordingly, the positions of the suction portion 2 and the discharge portion 4 may be set to have a positional relationship opposite to a positional relationship shown in FIG. 2 or set to have a positional relationship different therefrom.

In addition, the guide plate 12 constituting the flow path 6 and other members constituting the flow path 6 may be formed of an ultraviolet transmitting material or an ultraviolet reflective material. Here, examples of the ultraviolet transmitting material include glass, quartz (SiO₂), sapphire (Al₂O₃), an amorphous fluorine-based resin such as polytetrafluoroethylene (PTFE), an acrylic resin, and the like. The ultraviolet reflective material has a diffuse transmittance of 1%/1 mm or more and 20%/1 mm or less and a total reflectivity in the ultraviolet region of 60%/1 mm or more and 99.9%/1 mm or less. The sum of the diffuse transmittance and the total reflectivity in the ultraviolet region is desirably 90%/1 mm or more. Such an ultraviolet reflective material may include at least one of a silver material, an aluminum material, PTFE, a silicone resin, quartz glass containing air bubbles having a size of 0.05 μm or more and 10 μm or less therein, local-crystallized quartz glass containing crystal grains having a size of 0.05 μm or more and 10 μm or less therein, an alumina sintered body in the form of crystal grains having a size of 0.05 μm or more and 10 μm or less, or a mullite sintered body in the form of crystal grains having a size of 0.05 μm or more and 10 μm or less.

When the silver material or the aluminum material is used, a thin film functioning as a coating may be provided on a surface to prevent oxidation of the surface. For the thin film in this case, an acrylic resin, quartz glass, PTFE, or the like may be used. A method of forming the thin film using PTFE includes vapor deposition, sputtering, or the like. In addition, an inner periphery of the housing 16 may also be formed of the ultraviolet transmitting material or the ultraviolet reflective material. Further, a film formed of a photocatalytic active material may be provided on a surface of the guide plate 12 and an inner surface of the housing 16. That is, active oxygen may be generated by irradiation of ultraviolet rays to perform the decreasing and eliminating of the toxic subject by performing sterilization, antiviral treatment, deodorization, purification of the air and water by decomposing an organochlorine compound, for example, formaldehyde. In addition, examples of photocatalytic active material include titanium oxide, tungsten oxide, and the like.

### Description of Ultraviolet Light Source 8

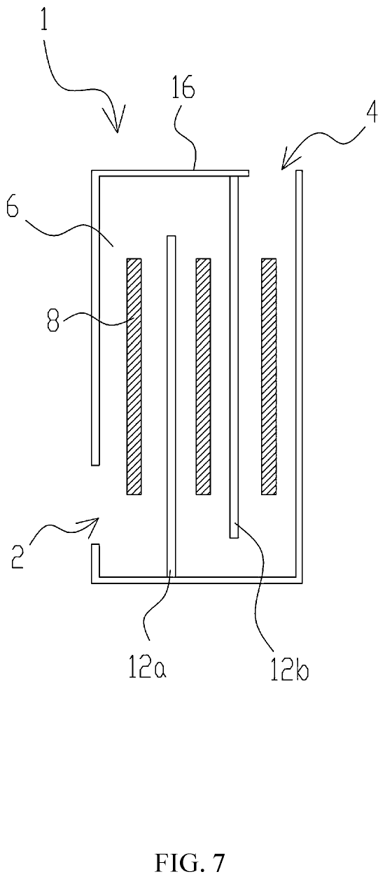

As long as the ultraviolet light source 8 may emit ultraviolet light into the flow path 6, a shape and arrangement thereof may be appropriately set. For example, the ultraviolet light source 8 may be in the form of a substantially elongated fluorescent tube (cylindrical tube). In addition, even in a case where the ultraviolet light source 8 in the form of the fluorescent tube is used, as shown in FIG. 7, the plurality of ultraviolet light sources 8 may be disposed in the flow path 6 so as to continuously emit the ultraviolet rays to the flowing fluid. Furthermore, even in a case where the ultraviolet light source 8 in the form of the fluorescent tube is used, the ultraviolet light source 8 may be used as a portion of the flow path 6. That is, the ultraviolet light source 8 is disposed along a flow direction, and may guide the fluid and emit the ultraviolet rays in a close range to the fluid.

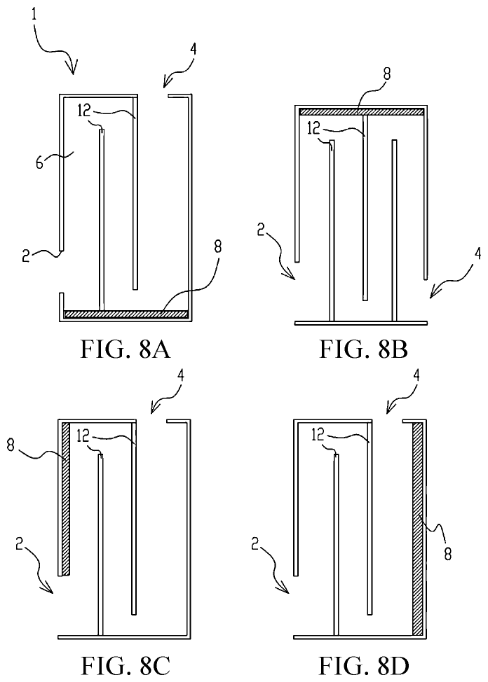

The ultraviolet light source 8 may be disposed at a position along a bottom portion as shown in FIG. 8A or at a position along an upper portion as shown in FIG. 8B. Even with such arrangement, the ultraviolet light source 8 may emit the ultraviolet rays in a wide range in the flow path 6. That is, since an emitting direction of the ultraviolet rays is parallel to a predetermined direction (e.g., a reciprocating direction) in the flow path 6, the ultraviolet rays are hardly blocked by the guide plate 12.

The ultraviolet light source 8 may be disposed in the vicinity of the suction portion 2 to extend in parallel to the reciprocating direction of the fluid as shown in FIG. 8C, and may be disposed in the vicinity of the discharge portion 4 to extend in parallel to the reciprocating direction of the fluid as shown in FIG. 8D. In this case as well, a length of the ultraviolet light source 8 in a longitudinal direction may be set according to a range in a height direction in which the flow path 6 in the housing 16 is formed.

However, when the ultraviolet light source 8 is disposed at the positions shown in FIGS. 8C and 8D, the ultraviolet rays are emitted in a direction non-parallel to the reciprocating direction of the fluid, and the ultraviolet rays are blocked by the guide plate 12. Therefore, it is desirable that the guide plate 12 is formed of the ultraviolet transmitting material. As a result, the ultraviolet rays may be transmitted through the guide plate 12 to emit substantially the entire area of the flow path 6. In addition, an inner surface 16a of the housing 16 may be formed of the ultraviolet reflective material so that the ultraviolet rays transmitted through the guide plate 12 are reflected. Accordingly, the ultraviolet rays may be reflected toward the flow path 6.

The ultraviolet light source 8 may also be disposed in a direction rotated by 90° in a plan view from the direction shown in FIG. 8A, in a direction in which the longitudinal direction is orthogonal to the height direction and a transverse direction. However, in this case, the ultraviolet light source 8 extends in a direction orthogonal to the flow of the fluid in the plan view, and accordingly, a region irradiated with the ultraviolet rays may be narrowed. Therefore, a concave reflector 18 is disposed to face the flow path 6 with the ultraviolet light source 8 interposed therebetween, thereby reflecting almost all the ultraviolet rays emitted to the concave reflector 18, to the flow path 6 side.

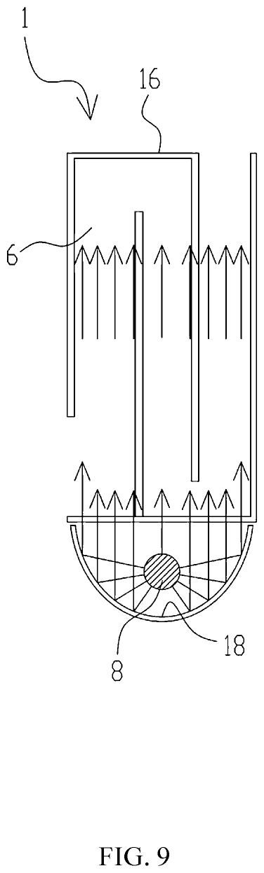

FIG. 9 is a diagram illustrating an example of arrangement of the concave reflector 18 for the ultraviolet light source 8. The concave reflector 18 has a concave reflective surface and the reflective surface is disposed to face the ultraviolet light source 8. More specifically, the concave reflector 18 is a curved mirror forming a portion of an elliptical shape, surrounds the ultraviolet light source 8, and is disposed to face the flow path 6 with the ultraviolet light source 8 interposed therebetween. The concave reflector 18 is positioned so that a focal point position of the ellipse formed by the reflective surface overlaps with the ultraviolet light source 8. According to the concave reflector 18 described above, the ultraviolet rays from the ultraviolet light source 8 may be converted into a parallel luminous flux and reflected.

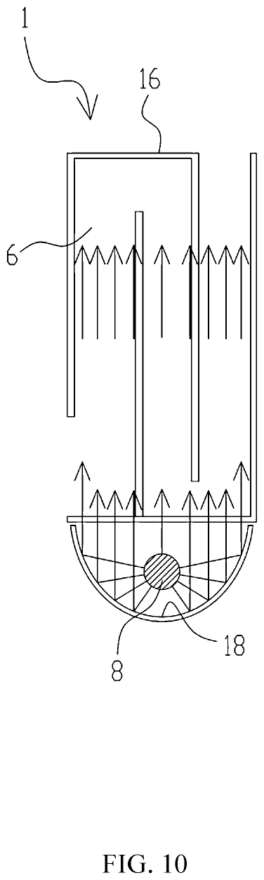

That is, as shown in FIG. 10, since the concave reflector 18 is set to face the flow path 6 with the ultraviolet light source 8 interposed therebetween, the ultraviolet rays directed to the outside of the flow path 6 which is the opposite side of the flow path 6, among the ultraviolet rays radially emitted from the ultraviolet light source 8, may be directed to the inside of the flow path 6. In addition, the reflected ultraviolet rays may become the parallel luminous flux and may be substantially parallel to the direction in which the fluid in the flow path 6 reciprocates. Accordingly, the ultraviolet rays reach an inner side of the flow path 6 in the reciprocating direction from the ultraviolet light source 8. In addition, as shown in FIG. 10, when the concave reflector 18 is positioned on a lower side of the flow path 6, an objective effective diameter or the like is set so as to face approximately the entire area of a lower end of the flow path 6.

In addition, in a case of the ultraviolet light source 8 in the form of a fluorescent lamp, the ultraviolet rays are radially emitted toward the entire peripheral portions from the ultraviolet light source 8, and accordingly, the ultraviolet rays may be emitted in a direction away from the flow path 6. Therefore, the ultraviolet rays directed to the outside of the flow path 6 may be forcibly directed to the direction of the flow path 6 by applying a paint for ultraviolet reflection to a portion of the fluorescent lamp.

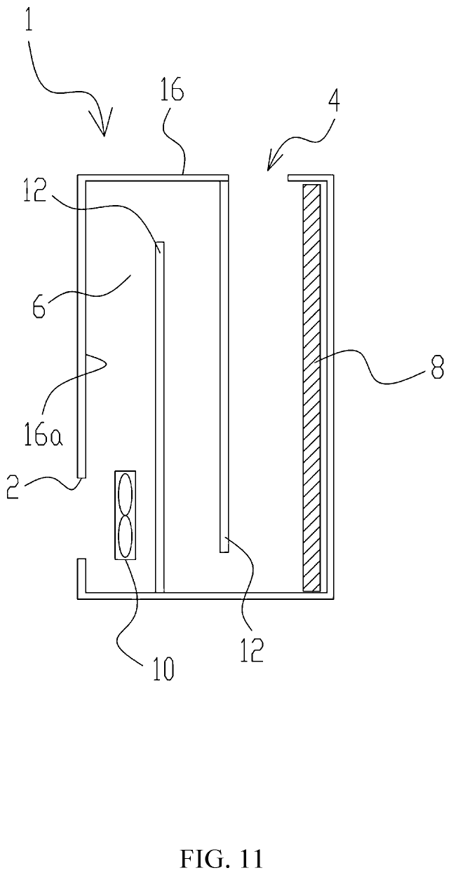

Next, the toxic subject decreasing and eliminating device having another configuration will be described with reference to FIG. 11. In the toxic subject decreasing and eliminating device shown in FIG. 11, the flow path 6 for reciprocating a fluid in the up and down direction is provided, the guide plate 12 is formed of the ultraviolet transmitting material, and the inner surface of the housing 16 is configured with an ultraviolet reflective surface. Also, the ultraviolet light source 8 is disposed to extend in the up and down direction along the inner surface of the housing 16 in the vicinity of the discharge portion 4 and to emit ultraviolet rays in a direction crossing the flow path 6.

In addition, the toxic subject decreasing and eliminating device 1 includes a flow generator 10 (the flow generating means) for generating a flow along the flow path 6 from the suction portion 2 side toward the discharge portion 4 side.

The flow generator 10 is disposed in the vicinity of the suction portion 2 in the flow path 6 and has a fan structure for causing the fluid to flow in the flow path 6. That is, the fan structure of the flow generating means 10 includes a propeller having a plurality of blades installed around a rotating shaft, a motor (driving source) for driving the propeller, and the like.

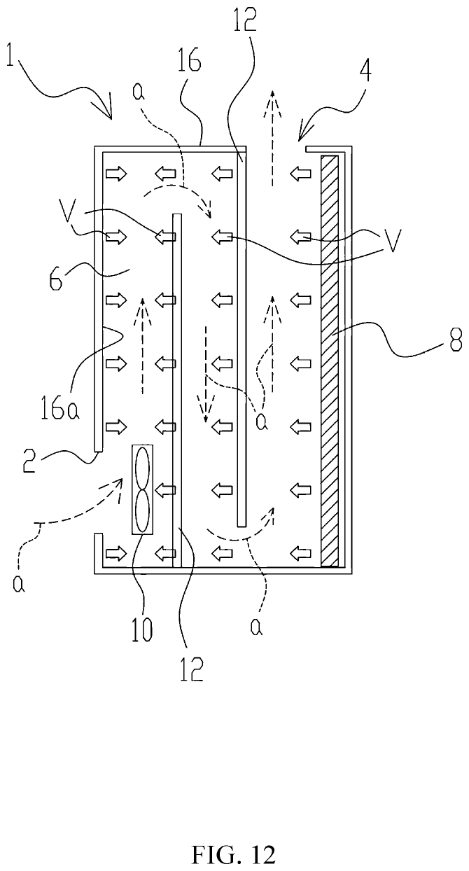

Herein, FIG. 12 is a diagram illustrating a flow direction of a fluid in the flow path 6 and a direction of ultraviolet rays. As the flow generator 10 is driven, the fluid outside the device is introduced to the flow path 6 through the suction portion 2. As shown with an arrow a of FIG. 12, the fluid reciprocates in the up and down direction and is discharged to the outside from the discharge portion 4. That is, the direction of the flow is guided by the guide plate 12, and the fluid moves to the discharge portion 4 and is discharged to the outside.

As shown with an arrow V of FIG. 12, the ultraviolet rays from the ultraviolet light source 8 are transmitted through the guide plate 12 and emitted to the inner surface 16a. That is, the ultraviolet rays from the ultraviolet light source 8 are emitted over approximately the entire portion of the flow path 6. In addition, since the inner surface 16a reflects the ultraviolet rays, the reflected ultraviolet rays are emitted to the inside of the flow path 6 again. Particularly, the ultraviolet rays reflected by the inner surface 16a at a position facing the ultraviolet light source 8 are directly directed toward the ultraviolet light source 8 side, and thus, the fluid in the flow path 6 may be irradiated with the ultraviolet rays from both sides of the ultraviolet light source 8 and the inner surface 16a.

As described above, since the ultraviolet rays are emitted to the inside of the flow path 6 and the guide plate 12 of the flow path 6 transmits the ultraviolet rays, the toxic subject in the fluid may be constantly exposed to the ultraviolet rays and the toxic subject may be decreased and eliminated reliably while the fluid flows through the flow path 6 and is discharged from the discharge portion 4.

In addition, in the flow path 6, since the fluid reciprocates in the up and down direction, the fluid moves within the flow path 6 for a longer time than the fluid moving linearly from the suction portion 2 to the discharge portion 4. Therefore, the time for the toxic subject to be exposed to the ultraviolet rays may be increased, thereby performing more reliable decreasing and eliminating. In addition, since the fluid flows down in a reciprocating manner, the toxic subject (particularly microorganisms) is forced to change its posture when the flow direction is changed, and as a result, the entire portion of the toxic subject may be irradiated with the ultraviolet rays in various directions. This may also make it possible to enhance decreasing and eliminating efficiency of the toxic subject.

In addition, since the inner surface 16a of the housing 16 has the ultraviolet reflectivity, the inner surface 16a reflects the ultraviolet rays emitted to the flow path 6 from the ultraviolet light source 8 to be emitted to the inside of the flow path 6 again. That is, since an ultraviolet reflecting means is positioned on a portion (the inner surface 16a) facing the ultraviolet light source 8 with the flow path 6 interposed therebetween, the ultraviolet rays emitted from the ultraviolet light source 8 and transmitted through the flow path 6 are reflected toward the flow path 6. Here, the amount of radiated ultraviolet rays for decreasing and eliminating the toxic subject may be expressed by irradiance (W/m²)×irradiation time (seconds). In the present disclosure, since the ultraviolet rays are reflected by the inner surface 16a to be emitted to the inside of the flow path 6, the toxic subject may be irradiated with the ultraviolet rays from both sides of the ultraviolet light source 8 and the inner surface 16a as if the toxic subject is interposed therebetween. Therefore, the irradiance of the ultraviolet rays may be improved, that is, a space density of the ultraviolet rays may be improved. As a result, the irradiation time until a predetermined amount of radiated ultraviolet rays is emitted to the toxic subject may be shortened and the decreasing and eliminating efficiency may be further improved.

In addition, any fan structure may be applied to the flow generator 10 as long as it may flow the fluid. For example, an axial-flow fan (a propeller fan), a mixed flow fan, a centrifugal fan (a multiblade fan, sirocco fan, radial fan, plate fan, turbo fan, limit load fan, airfoil fan, etc.), a centrifugal axial-flow fan, a vortex-flow fan, tangential fan (a cross-flow fan, etc.) may be applied.

The axial-flow fan which may be used in the flow generator 10 may be a double reversing type fan including two propellers spaced apart in an axial direction and rotating in opposite directions to each other.

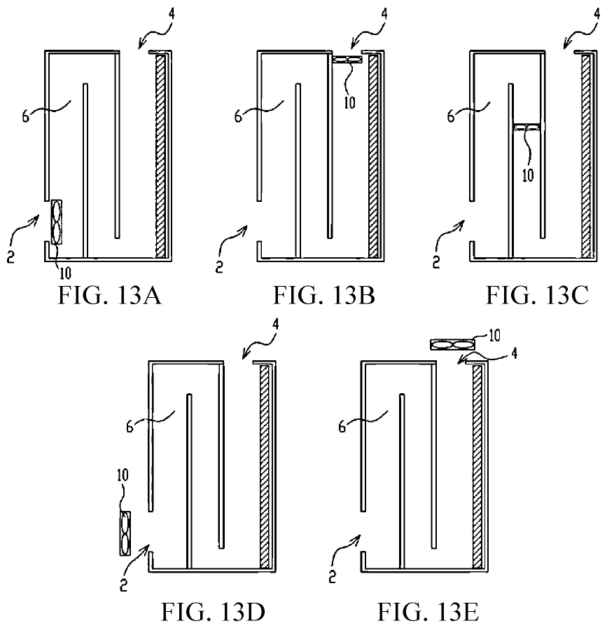

The position of the flow generator 10 may be appropriately set. For example, the flow generator 10 may be disposed in the vicinity of the suction portion 2 in the flow path 6 as shown in FIG. 13A, in the middle of the flow path 6 as shown in FIG. 13C, in the vicinity of the discharge portion 4 in the flow path 6 as shown in FIG. 13B, in the vicinity of the suction portion 2 in the outside of the flow path 6 as shown in FIG. 13D, or in the vicinity of the discharge portion 4 in the outside of the flow path 6 as shown in FIG. 13E.

Also, the flow generators 10 may be disposed at a plurality of portions, such as in the vicinity of the suction portion 2 and in the vicinity of the discharge portion 4 in the flow path 6. The flow generator 10 may also be disposed at a plurality of portions, such as in the vicinity of the suction portion 2 and in the vicinity of the discharge portion 4 outside the flow path 6, and the flow generators 10 may also be disposed inside and outside the flow path 6, respectively.

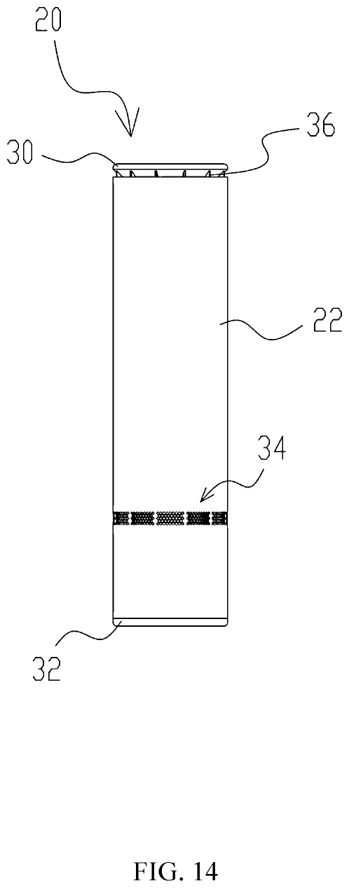

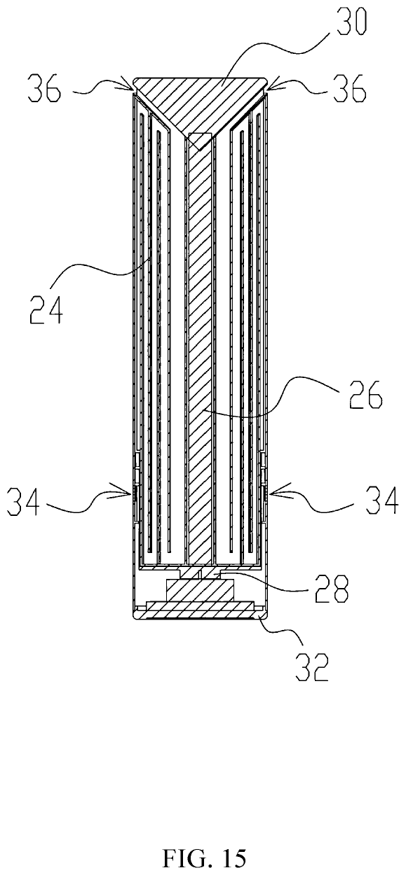

Next, another example of the toxic subject decreasing and eliminating device will be described with reference to FIG. 14. FIG. 14 is a diagram illustrating an exterior of a toxic subject decreasing and eliminating device 20 and FIG. 15 is a cross-sectional view illustrating the toxic subject decreasing and eliminating device 20. The toxic subject decreasing and eliminating device 20 is installed in a space where people gather, inhales the exhaled air or air as a fluid containing the exhaled air, and discharges the air by decreasing and eliminating a toxic subject in the inhaled air. In addition, the toxic subject decreasing and eliminating device 20 includes a plurality of suction portions 34 for making it possible to inhale the exhaled air from a plurality of directions, a flow path portion 24, and a discharge portion 36.

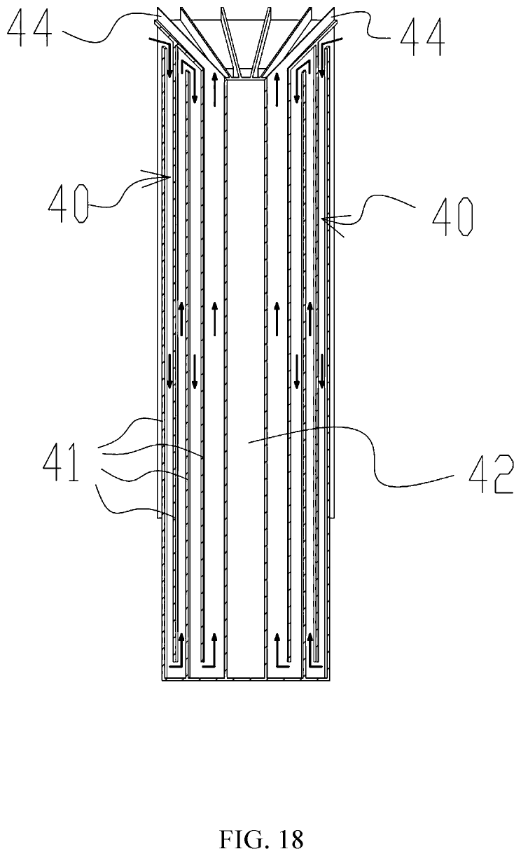

The toxic subject decreasing and eliminating device 20 includes a housing 22 having an approximately cylindrical shape. The flow path portion 24, an ultraviolet light source 26, and a blower 28 (the flow generating means) are disposed in the housing 22. In addition, a ceiling 30 is disposed on a ceiling portion of the housing 22 and a bottom portion 32 is provided on the bottom thereof. The axial direction of the housing 22 is set to be approximately parallel to the up and down direction, but is not particularly limited as long as the flow path portion 24 capable of holding the toxic subject for a sufficient time to cause the extinction of the toxic subject such as pathogenic microorganisms contained in the inhaled air. For example, the function described above may also be performed by providing an air flow path 40 (see FIG. 18) that is longer than a straight line distance from the suction portion 34 to the discharge portion 36.

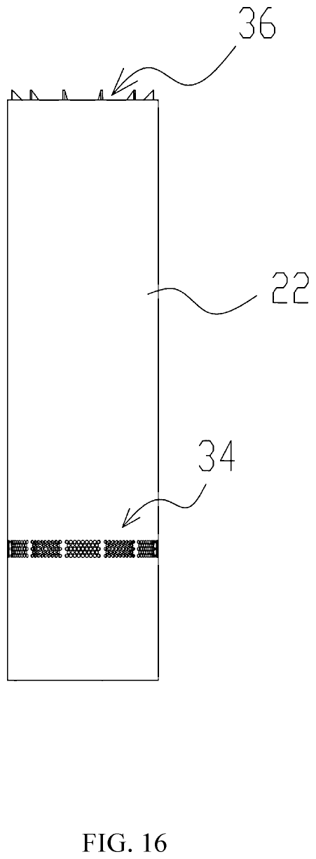

FIG. 16 is a front view illustrating the housing 22. The housing 22 is disposed with one end and the other end at an interval in the up and down direction. In addition, the housing 22 includes the plurality of suction portions 34 at an appropriately high position of an outer circumferential surface and the discharge portion 36 on an upper end side. Specifically, the suction portion 34 is positioned in the vicinity of a lower end side, that is, a lower half portion from an appropriate middle portion of the outer periphery in a height direction to a lowermost portion. In addition, the inner surface of the housing 22 is configured to reflect the ultraviolet rays.

Further, the discharge portion 36 is positioned on an upper end of the outer periphery and set so that a discharge direction of the air is inclined upward by an inclination of 45° with respect to the vertical direction. The horizontal directions of the suction portion 34 and the discharge portion 36 are set to be the same direction, but the horizontal directions of the suction portion 34 and the discharge portion 36 may not coincide and the inclination angle of the discharge portion 36 is not limited to 45°.

In addition, the suction portion 34 and the discharge portion 36 may have the opposite positional relationship or may have different arrangements, but at least for the installation position of the toxic subject decreasing and eliminating device 20 in an installation target space, the suction portion 34 is positioned at a position where the exhaled or exhaust air of a person is likely to be accumulated. The position and/or exhaust direction of the discharge portion 36 may be appropriately set, but, for example, the position or exhaust direction of the discharge portion 36 may be set to perform the exhaust toward a suction region by the suction portion 34. Also, the position or exhaust direction of the discharge portion 36 may be set to perform the exhaust toward a region in the installation target space that does not negatively affect a person who inhales the air, for example, a region where there are no people, or the exhaust direction thereof may be adjustable.

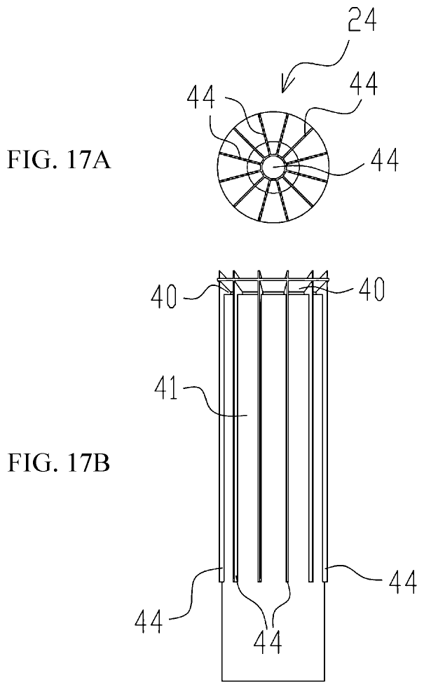

FIG. 17A is a plan view of an outer layer portion of the flow path portion 24 and FIG. 17B is a front view thereof. FIG. 18 is a cross-sectional view illustrating an inner portion of the flow path portion 24. The flow path portion 24 includes a plurality of air flow paths 40 in a member having an approximately cylindrical shape. In addition, the flow path portion 24 includes a plurality of guide plates 41 spaced apart in the radial direction, an installation space 42, and partition plates 44. The air flow path 40 is arranged in the circumferential direction to surround the installation space 42 and forms a flow path for holding the sucked air in the toxic subject decreasing and eliminating device 20 for a required time or longer while making the air flow down in a reciprocating manner in a direction parallel to the axial direction.

The required time herein is a time sufficient to sufficiently kill pathogenic microorganisms such as fungi and viruses attached to micro droplets or aerosols, which may be contained in inhaled air, by ultraviolet irradiation, or a time sufficient to decompose toxic molecules. This time is related to the amount of air flowing down per unit time, and this amount of air may be referred to as the amount of air to be inhaled per unit time from a suction port.

This inhalation amount is desirably set to be equal to or more than the amount of air exhaled by a person per unit time. That is, the inhalation amount is preferably set to at least 8 liters/min or more because the inhalation amount of a person per minute is 5 to 8 liters. In addition, the air is held in the air flow path 40 for the appropriate time and irradiated with the ultraviolet rays so that the sterilization is performed for 8 liters or more of the air flowing down in the air flow path 40 per minute by the ultraviolet irradiation.

The guide plate 41 is an annular plate-shaped member and arranged in plurality to be spaced apart concentrically in the air flow path 40. The guide plate 41 has an opening through which the air flows on one of upper and lower ends thereof, and makes the air flow to a downstream side (the discharge portion 36 side) through the opening.

In other words, as shown in FIG. 18, in the air flow path 40, the guide plates 41 in the form of an approximately concentric ring are arranged at intervals in a direction spaced apart from a shaft center, and each of an upper end or lower end of the guide plates 41 alternately communicates the adjacent air flow paths in the approximately concentric form. Accordingly, the sucked air is also displaced in a radial direction while being displaced in the reciprocating manner along the up and down direction so that the sucked air flows down toward the discharge portion 36.

The installation space 42 is a space formed in a central portion of the flow path portion 24 in this embodiment and the ultraviolet light source 26 is disposed therein. Accordingly, the installation space 42 functions as a portion of the flow path, as the ultraviolet light source 26 exists in the air flow path 40. The plurality of partition plates 44 divide the air flow path 40 along the circumferential direction. Specifically, the plurality of partition plates 44 are arranged at predetermined intervals along the circumferential direction so as to extend radially from the axial center of the flow path portion 24. Accordingly, the partition plates 44 define the space of each air flow path 40 in the circumferential direction.

As a result, each independent air flow path 40 is formed between the partition plates 44, and as shown with arrows of FIG. 18, the air entered each air flow path 40 gradually flows toward the inner side in the radial direction while reciprocating in the up and down direction from the outer side of the flow path portion 24 in the radial direction, and is discharged outside from the discharge portion 36 through the central portion of the flow path portion 24, that is, near the ultraviolet light source 26.

In addition, each of the partition plates 44 has one end (an upper end shown in FIG. 18) in a tapered shape in the axial direction of the flow path portion 24, protrudes from an upper end of the flow path portion 24, supports the ceiling 30, and functions as a portion of the discharge portion 36 to define the discharge direction of the air.

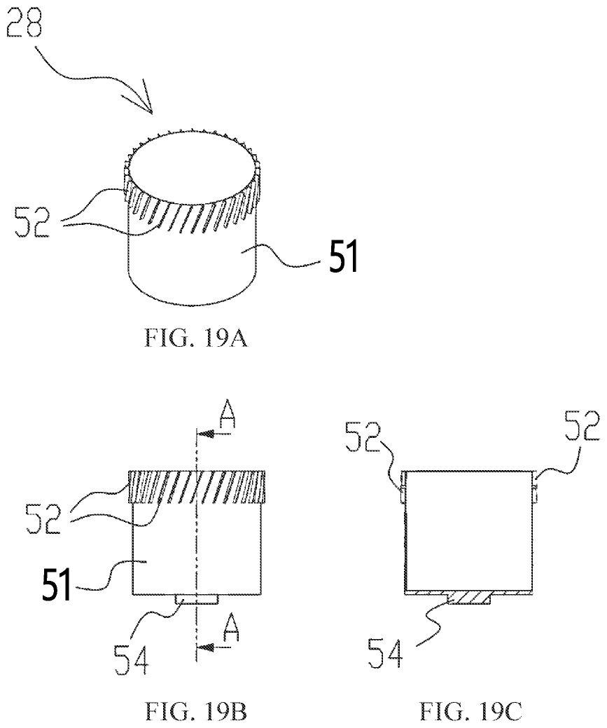

The blower 28 has a so-called propeller shape, and as shown in FIGS. 19A-19C, includes a rotating body 55 that rotates around a shaft center of the housing 22, a plurality of blades 52 formed on an outer circumferential surface of the rotating body 55, and a driving transmitter 54, etc. The rotating body 55 has a tubular shape capable of enclosing the flow path portion 24 in the housing 22. The wing or blade 52 is disposed between the suction portion 34 and the guide plate 41. The blade 52 generates the flow of the fluid flowing from the suction portion 34 to the discharge portion 36 through the air flow path 40 by the rotation. The driving transmitter 54 is formed on one end of the rotating body 55 and transmits the driving from a motor (not shown) to the rotating body 55.

In addition, the amount of the inhalation through the suction portion 34 by the flow generated by the blower 28 is appropriately set according to an installation environment or the like of the toxic subject decreasing and eliminating device 20. For example, when the toxic subject decreasing and eliminating device 20 is installed in a room where people gather, such as an office, the inhalation amount may be set to correspond to a total inhalation amount of the maximum number of people in the room or the number of people present around the eliminating device 20. Therefore, in a case of setting the inhalation amount capable of sucking approximately all of the air exhaled by four people, a rotation number of the rotating body 55 and a size or shape of the blade 52 are set so that the inhalation amount is 20 to 32 L (may also be 32 L or more).

The ceiling 30 is formed in an approximately circular cone shape and is supported by the partition plates 44 by bringing an inclined surface or tip end portion of the cone into contact with the partition plates 44. Accordingly, the discharge portion 36 is formed on the other end side of the housing 22. That is, since the one end portion of the partition plate 44 protrudes from the state of the flow path portion 24 and the ceiling 30 is supported by the one end of the partition plate 44, a gap is formed between the ceiling 30 and the discharge portion 36. Accordingly, the air flowing through the air flow path 40 is exhausted from the discharge portion 36 at an angle along the inclined surface of the ceiling 30.

The bottom portion 32 is detachably installed so as to block a lower portion of the housing 22, and for example, a driving motor or battery (not shown) for operating the blower 28 may be disposed on the lower portion of the housing 22 by detaching the bottom portion 32.

According to the toxic subject decreasing and eliminating device 20, the air containing the toxic subject may be introduced into the air flow path 40 through the suction portion 34 as the blower 28 is driven. The air introduced into the air flow path 40 gradually flows down toward the center of the flow path portion 24 in the radial direction while reciprocating in the up and down direction, and is discharged from the discharge portion 36.

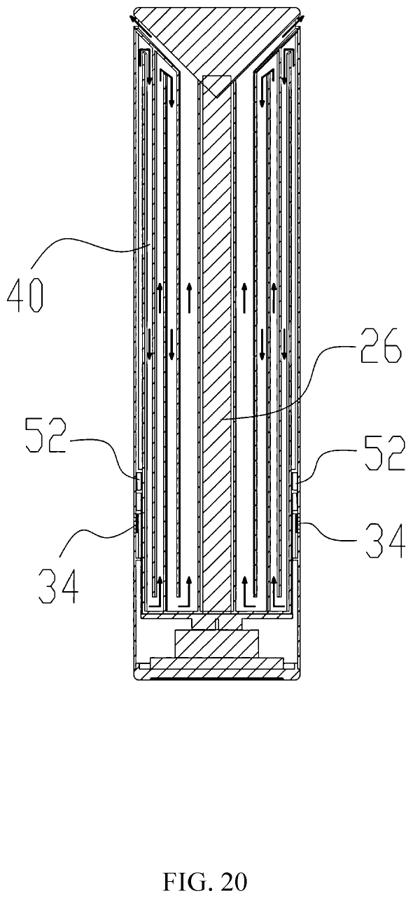

In addition, since the plurality of the air flow paths 40 are formed, the air from the plurality of directions may be taken in and discharged at the same time. In this case, in a plan view, the air sucked from one direction through the suction portion 34 is discharged toward the one direction through the discharge portion 36. That is, as shown in FIG. 20, on the left side of the toxic subject decreasing and eliminating device 20, the air taken from the suction portion 34 facing the left side is caused to flow from the outer side to the inner side in the radial direction while reciprocating in the up and down direction and is discharged toward the left side from the discharge portion 36.

Similarly, on the right side of the toxic subject decreasing and eliminating device 20, the air taken from the suction portion 34 facing the right side is caused to flow from the outer side to the inner side in the radial direction while reciprocating in the up and down direction and is discharged toward the right side from the discharge portion 36.

In this case, the discharge portion 36 is provided on an uppermost portion of the housing 22 and defined by an upper end of the air flow path 40 on an innermost layer, that is, closest to a shaft center, and a lower surface of the ceiling 30 in an inverse conical shape. Accordingly, the air discharged from the discharge portion 36 is blown out diagonally upwards radially.

Therefore, the air discharged diagonally upwards from the discharge portion 36 which is disposed sufficiently higher than the suction portion 34 which is disposed in the vicinity of a height position of the respiratory tract, such as the oral cavity or the nasal cavity, where exhaled air and exhaust air of people tend to be accumulated, may discharge the air toward a portion higher than the oral cavity or the nasal cavity and may suppress disturbing the air flow in the area where exhaled air and exhaust air of people tend to be accumulated.

In addition, approximately the entire area inside the device may be irradiated with the ultraviolet rays by the ultraviolet light source 26. That is, the air flowing through the air flow path 40 is irradiated with the ultraviolet rays by the ultraviolet light source 26 and the ultraviolet rays are transmitted through the guide plates 41 and reflected by the inner surface of the housing 22 while spreading radially.

Therefore, even if the toxic subject contained in the air is covered with dust or the like, the hidden toxic subject may be decreased and eliminated, since the ultraviolet rays may be emitted to approximately the entire portion in various directions.

As described above, the toxic subject in the air may also be decreased and eliminated by the toxic subject decreasing and eliminating device 20. That is, the emitted ultraviolet rays are transmitted through the guide plates 41, spread over the entire area of the flow path portion 24, and are constantly emitted to the toxic subject in the air flowing through the air flow path 40. Therefore, the toxic subject contained in the air may be substantially decreased and eliminated until the toxic subject reaches the discharge portion 36. In addition, when the air in a region where the toxic subject is likely to exist is sucked by the suction portion 34, the air in the suction region is sucked partially, the toxic subject contained in the sucked air is reliably decreased and eliminated, and the air after the decreasing and eliminating is discharged to a space, which is a region different from the suction region and is a region where the toxic subject is less likely to exist. Accordingly, the toxic subject existing in the space may be gradually and reliably decreased and eliminated substantially without agitating the air in the space.

In addition, the ultraviolet rays transmitted through the flow path portion 24 are reflected by the inner surface of the housing 22 and emitted to the inside of the flow path portion 24 again, and thus the ultraviolet rays may be emitted in the plurality of directions with an increase in the amount of ultraviolet rays emitted to the toxic subject in the air flow path 40. Also, the flowing toxic subject flows down in the reciprocating manner in the up and down direction and a direction thereof is not even determined. Accordingly, the entire portion of the toxic subject may be irradiated with the ultraviolet rays, which makes it possible to improve the decreasing and eliminating efficiency of the toxic subject by the ultraviolet rays.

In addition, since the air flow path 40 has a path reciprocating along the axial direction, a distance for the toxic subject moving through the air flow path 40 becomes longer and a time during which the toxic subject flows becomes longer. Since this also leads to an increase in the amount of ultraviolet rays emitted to the toxic subject, the decreasing and eliminating efficiency may be improved.

In addition, since the plurality of air flow paths 40 are arranged in the circumferential direction, the decreasing and eliminating of the toxic subject around the toxic subject decreasing and eliminating device 20 may be performed. Also, since the air sucked from one direction on the plane is discharged toward the one direction on the plane, in a case where, for example, the exhaled air of a person infected with a virus is sucked, the air containing the exhaled air is discharged toward the person. Therefore, the air containing the exhaled air of the virus-infected person is not directed to a person other than the infected person, in addition to the inactivating of the virus through the air flow path 40. Thus, a sense of security may be provided without making individuals experience anxiety due to virus infection or the like from other person.

The direction of the housing 22 is not limited thereto and the housing 22 may be placed horizontally, that is, may be disposed so that the axial direction is the horizontal direction.

In addition, although the inner surface of the housing 22 is formed of the ultraviolet reflective material, the guide plate 41 positioned on an outermost layer of the flow path portion 24, that is, on an outermost side in the radial direction may be formed of the ultraviolet reflective material. Alternatively, a surface of the corresponding guide plate 41 facing the ultraviolet light source may be provided with ultraviolet reflectivity.

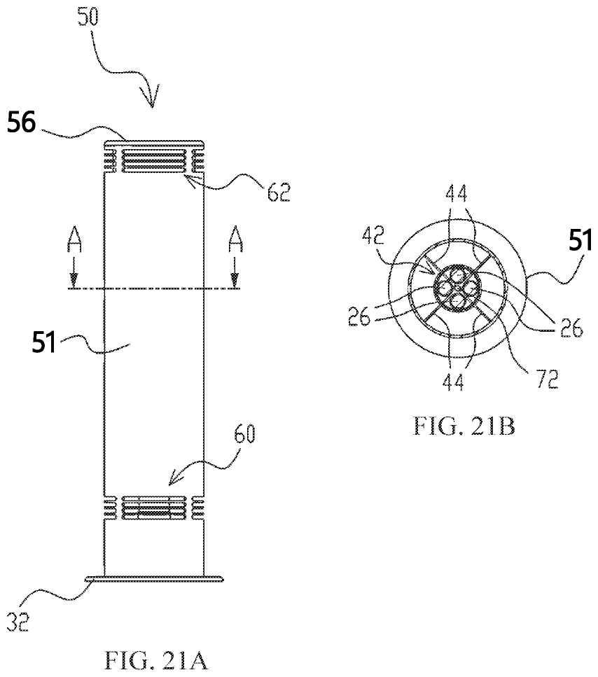

Next, a toxic subject decreasing and eliminating device 50 having another configuration will be described. The same components as those described above will be described with the same reference numerals. FIG. 21A is a diagram illustrating an exterior of the toxic subject decreasing and eliminating device 50 and FIG. 21B is a cross-sectional view taken along line A-A of FIG. 21A. The toxic subject decreasing and eliminating device 50 includes a housing 51 having an approximately cylindrical shape. A suction portion 60 is directly formed on a lower end portion of an outer periphery of the housing 51 and a discharge portion 62 is directly formed on an upper end portion of the outer periphery thereof. Also, an upper end opening of the housing 51 for inserting the ultraviolet light source 26 is blocked by a ceiling 56.

In addition, as shown in FIG. 21B, an inner portion of the housing 51 is divided into four sections in the circumferential direction by the partition plates 44. That is, four air flow paths 40 are arranged in the circumferential direction in the housing 51. In addition, four ultraviolet light sources 26 are inserted into the installation space 42 and a position of each of the ultraviolet light sources 26 in the installation space 42 is set so as to be paired with each of the air flow paths 40.

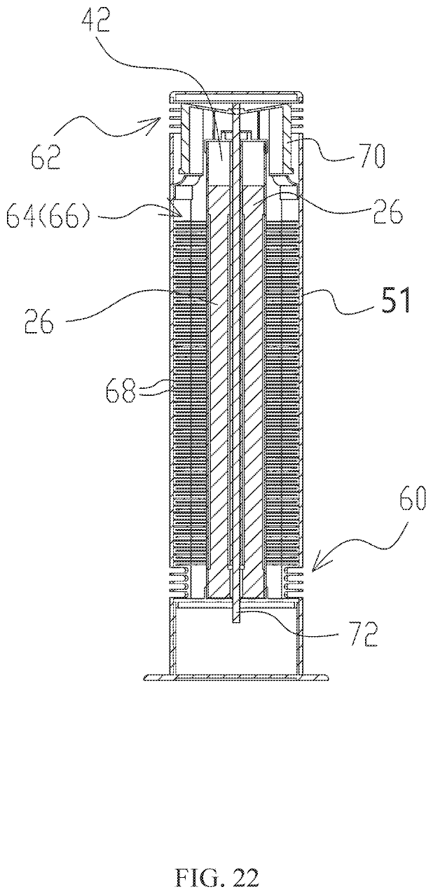

Further, in the installation space 42, a rotating shaft 72 of a blower 70 (see FIG. 22) is disposed to coincide with a position where the ultraviolet rays from each ultraviolet light source 26 are not obstructed, that is, the shaft center of the housing 51.

FIG. 22 is a cross-sectional view illustrating the toxic subject decreasing and eliminating device 50. A flow path portion 64, the blower 70, and the like are disposed in the housing 51. The flow path portion 64 defines an air flow path 66 through which the fluid gradually flows down along the axial direction while reciprocating in the radial direction. Specifically, in the housing 51, a plurality of plate-shaped guide plates 68 is disposed along the axial direction with surfaces thereof facing the orthogonal direction.

The guide plate 68 has an opening on an outer or inner side in the radial direction of the housing 51. Specifically, an opening for communicating adjacent spaces in the axial direction which are partitioned by the guide plates 68 is formed on the guide plate 68. The positions of the openings are set so that an inner side in the radial direction and an outer side in the radial direction of the adjacent guide plates 68 are alternately aligned.

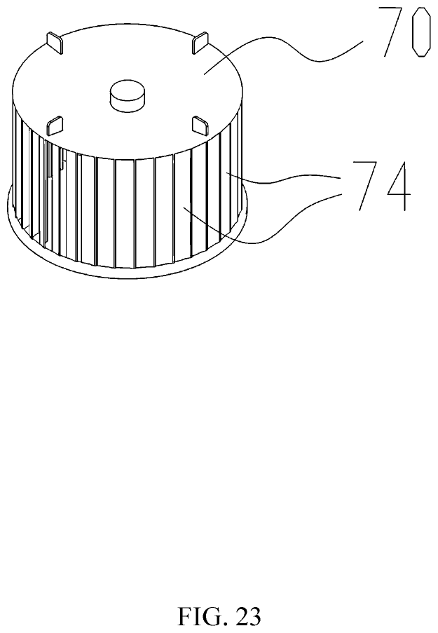

FIG. 23 is a diagram illustrating the blower 70. The blower 70 has a centrifugal fan structure formed by aligning a plurality of vertically elongated plate-shaped blades 74 in the tubular form, and a rotating shaft thereof is disposed in the vicinity of the discharge portion 62 to coincide with the axial direction of the housing 51. In addition, the blower 70 is fixed to one end of the rotating shaft 72 extending in the axial direction. The other end of the rotating shaft 72 is connected to a motor (not shown) disposed on the side of the bottom portion 32.

According to such a configuration, the ultraviolet rays may be emitted to the space between the guide plates 68 directly from the ultraviolet light source 26, and accordingly, approximately the entire area of the air flow path 66 may be irradiated with the ultraviolet rays without forming the guide plates with the ultraviolet transmitting material.

It is described that the four ultraviolet light sources are disposed in the toxic subject decreasing and eliminating device 50, and since the ultraviolet light sources emit the ultraviolet rays radially, the rotating shaft 72 may be irradiated with the ultraviolet rays. Therefore, an ultraviolet reflective surface may be provided on a portion of a surface (or an inner surface) of a fluorescent tube of the ultraviolet light source by using an ultraviolet reflective paint or the like, thereby making all the ultraviolet rays emitted to the rotating shaft 72 side be directed toward the air flow path side. By doing so, the amount of ultraviolet rays in the air flow path may be increased and the decreasing and eliminating property of the toxic subject may be improved.

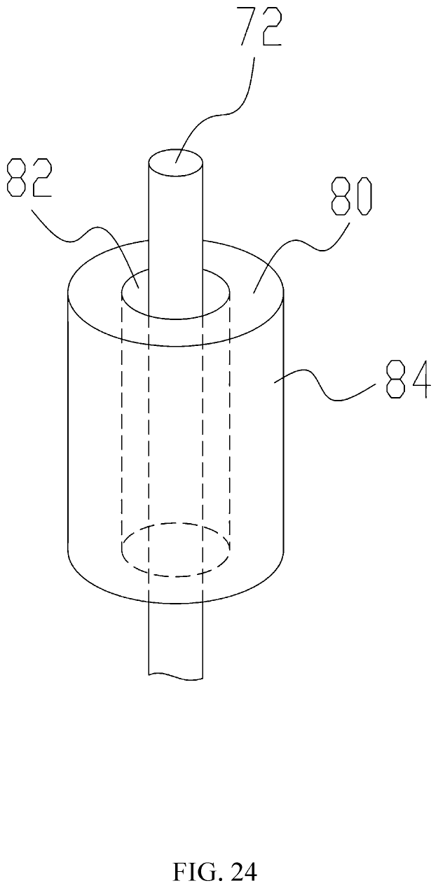

In addition, although the four ultraviolet light sources 26 and the rotating shaft 72 are arranged in the installation space 42, one ultraviolet light source and the rotating shaft 72 may be disposed therein. In this case, the ultraviolet light source is formed to have a shape of a fluorescent tube having a torus-shaped cross section so that the rotating shaft 72 is inserted into a central cavity. That is, as shown in FIG. 24, a columnar ultraviolet light source 80 having an inner annular surface 82 and an outer annular surface 84 spaced apart in the radial direction is provided and performs the ultraviolet light emission between the inner annular surface 82 and the outer annular surface 84. By doing so, a space may be formed on the shaft center side with respect to the inner annular surface 82 and the rotating shaft 72 may be disposed in the space.

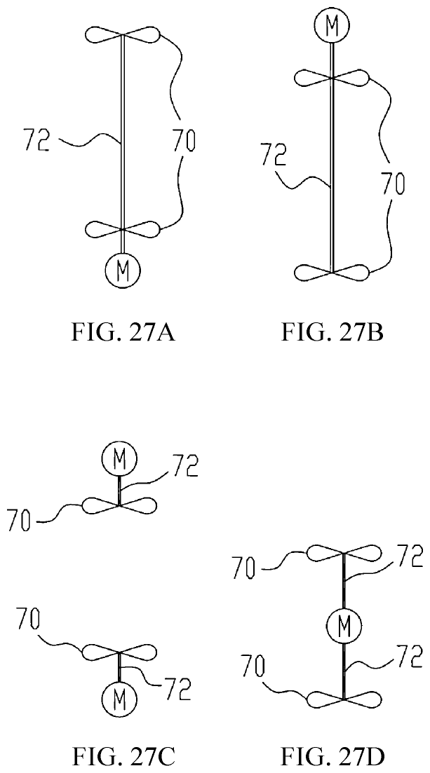

In addition, although it is described that the blower 70 is disposed on the one end of the rotating shaft 72, a plurality of blowers 70 may be fixed to a single rotating shaft 72. For example, as shown in FIG. 27A, the blowers 70 may be disposed on one end and a middle portion of the rotating shaft 72, respectively, or as shown in FIG. 27B, a driving motor M may be connected to the one end (an upper portion) of the rotating shaft 72 and the blowers 70 may be disposed on the other end and the middle portion thereof, respectively. In this way, both blowers 70 may be driven by rotating the rotating shaft 72 with one driving motor M. Also, the driving motor M and the rotating shaft 72 may be disposed for each blower 70 (see FIG. 27C), or two rotating shafts 72 may be connected to a double-shaft motor and each blower 70 may be fixed to and disposed on each rotating shaft 72 (see FIG. 27D).

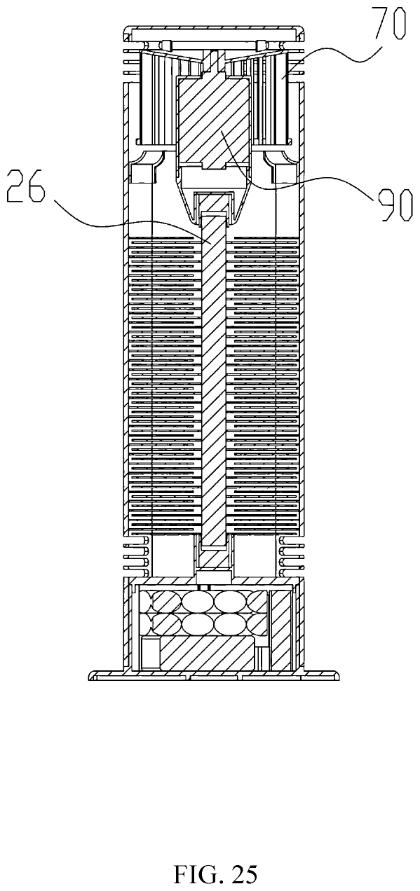

The toxic subject decreasing and eliminating device 50 may be configured such that the rotating shaft is not inserted into the installation space 42. For example, as shown in FIG. 25, the motor 90 for driving the fan structure may be disposed at a position adjacent to the blower 70 outside the installation space 42.

In addition, a filter for collecting foreign matters may be provided in the toxic subject decreasing and eliminating device. That is, when the fluid is taken in, foreign matters such as dust may be mixed with the fluid and the foreign matters may be accumulated in the suction portion or in the middle of the flow path. Accordingly, the foreign matters may be collected by providing a filter. It is desirable that the filter is disposed to be detachable so that it may be replaced when it is clogged.

Also, in a case of collecting dust in the air, the filter may be, for example, a pre-filter for mainly collecting particles having a size of 50 μm or more, a medium efficiency particulate air (MEPA) filter for mainly collecting particles having a size of 25 μm or more, a high efficiency particulate air (HEPA) filter for collecting particles having a size of 0.3 μm, an ultra-low particulate air (ULPA) filter for collecting particles having a size of 0.15 μm.

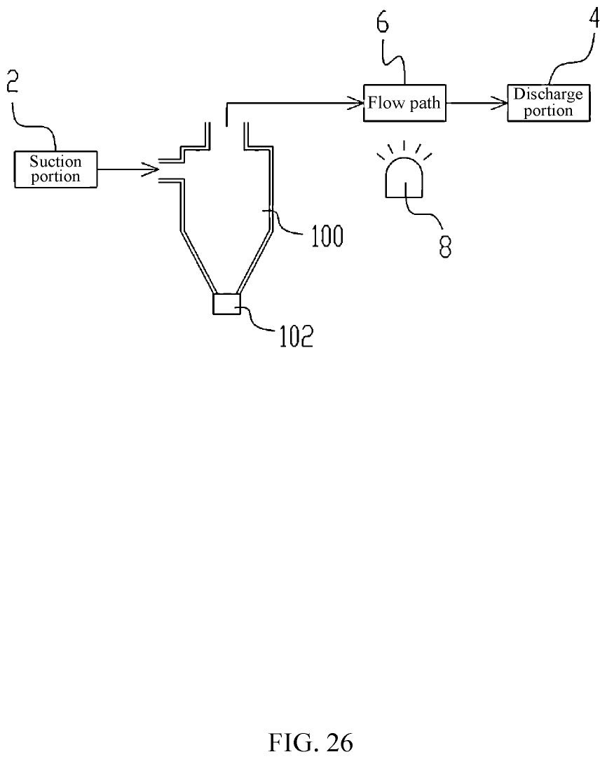

In addition, a cyclone chamber for separating the dust in the air by using powder separation by cyclone may be provided. That is, as shown in the drawing illustrating the schematic configuration of FIG. 26, a cyclone chamber 100 has an inverse conical shape, includes a dust collecting portion 102 for collecting dust on a bottom portion, and is disposed between the suction portion 2 and the flow path 6. Accordingly, the air flowing through the suction portion 2 first stays in the cyclone chamber 100 in a vortex manner and then flows to the flow path 6. At this time, the dust comes into contact with an inner circumferential surface of the cyclone chamber 100 and falls into and is accumulated in the dust collecting portion 102. Therefore, the dust may be separated from the air.

In addition, in the cyclone chamber, an integral and/or separate light source corresponding to the ultraviolet light source may be disposed in the cyclone chamber or the like to emit ultraviolet rays, or the cyclone chamber may be formed of the ultraviolet transmitting material or the like so that the ultraviolet rays are emitted directly from the ultraviolet light source.

In addition, the flow path may be set so that each cross-sectional area of each space sandwiched between the guide plates is the same. That is, the flow path may be set to have the same cross-sectional area for each region partitioned by the guide plates provided from the upstream side to the downstream side of the flow path in the flow direction. By doing so, a flow velocity of the fluid in the suction portion may be set to be substantially the same as a flow velocity thereof in the discharge portion.

In addition, the flow path may be set so that the cross-sectional area of each space sandwiched between the guide plates is reduced toward the downstream side in the flow direction. That is, the flow path may be set so that the cross-sectional area of each region partitioned by the guide plates is gradually reduced toward the downstream side. In this way, the flow velocity of the fluid flowing down through the flow path may be configured to gradually increase, and therefore, the fluid may be discharged from the discharge portion at a flow velocity faster than a flow velocity in the suction portion.

The flow path may be set so that the cross-sectional area of each space sandwiched between the guide plates expands toward the downstream side in the flow direction. That is, the flow path may be set so that the cross-sectional area of each region partitioned by the guide plates gradually expands toward the downstream side. In this way, the flow velocity of the fluid flowing down through the flow path may be configured to gradually decrease, and therefore, the fluid may be discharged from the discharge portion at a flow velocity slower than a flow velocity in the suction portion. When the flow velocity is decreased, the disturbance of the surrounding airflow due to the discharge of the fluid may be suppressed.

An area of the discharge port for the fluid in the discharge portion may be set to be larger than an area of the suction port for the fluid in the suction portion. By doing so, the flow velocity in the discharge portion may be set to be slower than the flow velocity in the suction portion. As described above, when the size of the openings, such as the suction port in the suction portion and the discharge port in the discharge portion is changed, the flow path may be set so that the suction portion performs high-speed suction and the discharge portion performs low-speed discharge.

In addition, when the area of the discharge port for the fluid in the discharge portion is set to be smaller than the area of the suction port for the fluid in the suction portion, the flow path may be set so that the suction portion performs low-speed suction and the discharge portion performs high-speed discharge.

In addition, a shape of the suction port capable of sucking the fluid in the suction portion may be appropriately set. For example, the suction portion may include a suction port having an extended opening so as to suck the fluid from a wide area. Also, the suction portion may have a nozzle shape for sucking the fluid in a single direction or a suction port having an opening that becomes narrow along the flow direction. By providing the nozzle shape or the suction port that becomes narrow along the flow direction, the sucked fluid may flow down to the flow path as a jet flow.

In addition, a shape of the discharge port capable of discharging the fluid in the discharge portion may be appropriately set. For example, the discharge portion may include a discharge port having an extended opening so as to discharge the fluid to a wide area. Also, the discharge portion may have a nozzle shape for discharging the fluid in a single direction or a discharge port having an opening that becomes narrow along the flow direction.

The discharge portion may include a continuous or intermittent exhaust port extending toward one direction so that an air curtain is generated by the exhaust of the air as the fluid from the exhaust port. In addition, the discharge portion may be set to discharge a jet flow.

In addition, in a case of using the toxic subject decreasing and eliminating device of the present disclosure for the purpose of inactivating or sterilizing pathogenic microorganisms in the air by taking in the ambient air, the toxic subject decreasing and eliminating device may be installed in a space where people gather or a space that is likely to be crowded, for example, such as, for example, an office, conference room, restaurant, show room, library, school, kindergarten, nursery school, shop, entertainment facilities (karaoke, aquarium, planetarium, movie theater, art gallery, museum, bowling alley, etc.), vehicle (car, airplane, ship, or train), and the like.

In a case of using the toxic subject decreasing and eliminating device for the purpose of so-called purification of contaminated water for taking in a fluid as a liquid and performing the decreasing and eliminating of the toxic subject, the toxic subject decreasing and eliminating device may be installed in a plant, septic tank, piping, a connected portion between the pipes, etc.

The toxic subject decreasing and eliminating device of the present disclosure may be built in, incorporated in, or combined with separate equipment. The target equipment may be appropriately selected as long as at least a suction portion and a discharge portion communicate with the outside, and examples thereof may include a vehicle roof, seat backrest, seat headrest, concrete panel, air conditioner, vacuum cleaner, table, desk, chair, wall, elevator, etc. In particular, the toxic subject decreasing and eliminating device may be used to be embedded in equipment installed in the space where people gather or the space that is likely to be crowded.

The housing may be configured with a plurality of members and may be configured to be divided, for example, in the axial direction or circumferential direction. In addition, the housing and the flow path portion may be integrally formed, but the housing and the flow path portion may be separate portions.

Although the decreasing and eliminating of the toxic subject has been performed by the ultraviolet irradiation, a heating means for heating the inside of the flow path to an extent capable of decreasing and eliminating the toxic subject or an electric field generating means for decreasing and eliminating the toxic subject by locally causing a micro-discharge phenomenon or generating an electric field in the flow path for adsorbing the toxic subject (particularly, pathogenic microorganisms) to electrodes by a pair of positive and negative electrodes arranged to oppose each other may be provided. Also, the decreasing and eliminating of the toxic subject may be performed by disposing the heating means and/or electric field generating means instead of the ultraviolet light source.

In the housing, a partition that is integrated with the outer circumferential surface or that may be mounted on the outer circumferential surface may be disposed. By combining with the partition, a space around the device may be partitioned from the space where the people gather or the space that is likely to be crowded, and the air obtained by decreasing and eliminating the toxic subject may be prevented from being discharged to the outside of the partitioned space.

The toxic subject decreasing and eliminating device may further include at least one of a temperature sensor, a humidity sensor, a human sensor, or a contamination sensor, and may control the flow generating means based on the detection by the sensor. For example, when the sensor detects the presence of a person in a surrounding portion, the operation of the flow generating means may be performed. In addition, the flow generating means may be stopped when the sensor does not detect a person anymore, when a predetermined time has elapsed after the operation of the flow generating means has performed, or the like.

In the embodiment described above, it is described that the flow path has the ultraviolet transmitting property, but an ultraviolet high-order reflective flow path capable of reflecting ultraviolet rays multiple times inside may be formed. For example, a reflective layer formed of the ultraviolet reflective material is formed over substantially the entire inner circumferential surface of a spiral or vortex flow path. Accordingly, the ultraviolet rays from the ultraviolet light source are emitted to a wide range from the upstream side to the downstream side in the flow direction while being reflected multiple times (high-order reflection) within the flow path.

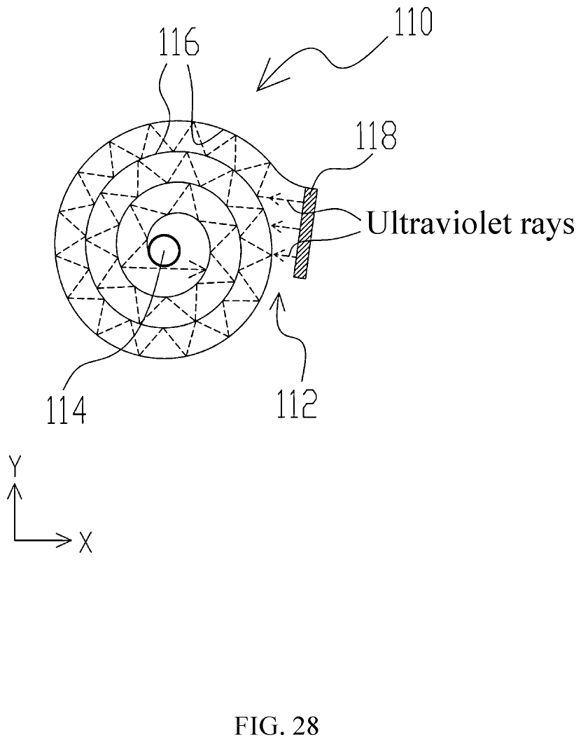

FIG. 28 is a diagram illustrating a vortex-shaped flow path 110, and the flow path 110 includes a suction portion 112 at an outermost portion of the flow and a discharge portion 114 at a central portion thereof. The flow path 110 includes a fluid guide surface (an ultraviolet reflective surface) 116 that defines a width of the flow path and has the ultraviolet reflectivity. In the vicinity of the suction portion 112 on the outermost portion of the flow path 110, an ultraviolet light source 118 is disposed to face the fluid guide surface 116. For the ultraviolet light source 118 at this time, a direction in which the ultraviolet rays are emitted or the like is set so that the ultraviolet rays are emitted to the facing fluid guide surface 116 and the downstream side in the flow direction.

When the horizontal direction is set as an X axis and the up and down direction is set as a Y axis in FIG. 28, the flow path 110 forms a vortex shape along a plane parallel to an XY plane. In addition, the discharge portion 114 disposed on the central portion of the flow path 110 is formed to discharge the fluid in a direction orthogonal to the XY plane.

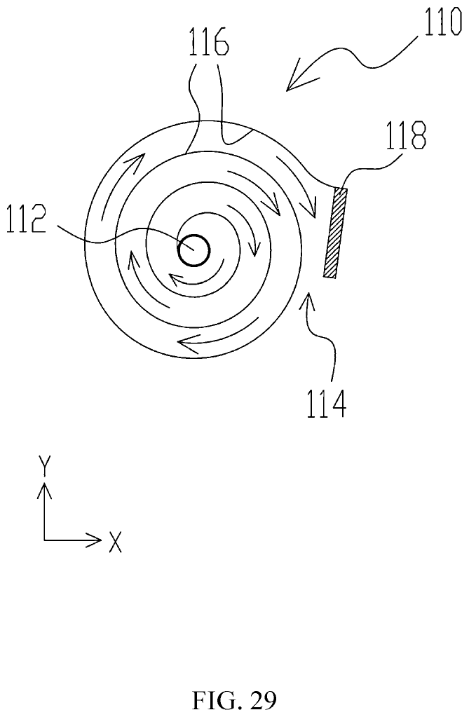

Also, the positions of the suction portion 112 and the discharge portion 114 may be interchanged. That is, as shown in FIG. 29, the suction portion 112 may be disposed on the central portion of the flow path 110 and the discharge portion 114 may be disposed on the outermost portion of the flowing flow path 110. A fan shape or installation position of the flow generator is not particularly limited and may be set appropriately. For example, the flow generator may be disposed in the vicinity of the suction portion 112 and/or discharge portion 114. Accordingly, when the suction portion 112 is disposed on the central portion of the flow path 110 shown in FIG. 29, a centrifugal fan-shaped flow generator may be disposed. That is, the flow generator is installed in the vicinity of the suction portion 112 in a direction in which a rotating shaft thereof is orthogonal to the XY plane. Accordingly, the fluid sucked in the direction orthogonal to the XY plane reaches the discharge portion 114 while flowing outward in the vortex manner along the flow path 110 from the suction portion 112, as shown with an arrow of FIG. 29.

The cross-sectional shape of the flow path is not particularly limited and may be appropriately set. The cross-sectional shape of the flow path may be, for example, a polygonal shape (a triangular shape, rectangular shape, pentagonal shape, hexagonal shape, etc.) or circular shape (a perfect circle shape, elliptical shape, oval shape, etc.) The circular shape is desirably used because more excellent flow state may be obtained.

A reflective layer of the fluid guide surface 116 may also be configured by providing, for example, a thin film of metal having ultraviolet reflectivity (silver, aluminum, nickel, copper, etc.) on a surface of the fluid guide surface 116. In addition, the reflective layer may extend continuously or intermittently on the fluid guide surface 116, as long as it is disposed on the fluid guide surface 116 to be provided over at least approximately the entire area of the flow path 110.

Next, a cumulative ultraviolet irradiation energy when the ultraviolet rays are reflected in the ultraviolet high-order reflective flow path will be described by using a case, where an aluminum thin film is used, as an example. Here, a reflectivity of ultraviolet rays by the aluminum thin film is 92.3%, which corresponds to a photon quantity decreased by 7.7% from the photon quantity before the reflection when the ultraviolet rays are reflected by the aluminum thin film. Accordingly, a residual capacity of photons (which is 100% when the number of reflections is 0) exponentially decreases as the residual capacity is 92.3% when the number of reflections of the ultraviolet rays by the aluminum thin film is 1, is 85.2% when the number of reflections is 2, and is 78.6% when the number of reflections is 3.

If such reflection is repeated, the residual capacity is approximately 0.1% when the number of reflections is 94, and the residual capacity is approximately 0% when the number of reflections is 95. In other words, the photons remain until the 94th reflection, and thus the cumulative ultraviolet irradiation energy may be considered as effective.

A value regarding the cumulative ultraviolet irradiation energy in a case where a flow path capable of performing the ultraviolet reflection 95 or more times is formed is estimated as below by using a relationship of the exponential function described above. In the following equation, the cumulative ultraviolet irradiation energy when the reflectivity of the ultraviolet rays is set as 92% (a reduction rate of photons at the time of reflection is set as 8%) and the ultraviolet rays are reflected ∞ times is calculated.

∫₀^{∞}e^{-0.08x}dx=12.5 Equation 1

Accordingly, a value of 12.5 is obtained as the cumulative ultraviolet energy when the ultraviolet rays are reflected ∞ times (after the 95th reflection, the residual capacity of photons is approximately 0% and the cumulative ultraviolet irradiation energy is effective until the 94th reflection). This indicates that the cumulative ultraviolet irradiation energy is generally equivalent to 12.5 times when the ultraviolet irradiation energy in a case where the ultraviolet rays are emitted once is set as 1.

Even the ultraviolet high-order reflective flow path substantially continuously emits the ultraviolet rays to the fluid flowing through the flow path 110, thereby improving the decreasing and eliminating efficiency by increasing the amount of ultraviolet rays radiated to the toxic subject. In addition, although the amount of photons of ultraviolet rays from the ultraviolet light source may gradually decrease with each reflection, the ultraviolet rays are reflected along the flow path, and therefore, a considerably larger amount of radiated ultraviolet rays (ultraviolet rays with the cumulative ultraviolet irradiation energy of 12.5 times at most) may be emitted to the toxic subject, compared to a case where the toxic subject is caused to pass through a portion where the ultraviolet rays are emitted to the inside of the flow path with no reflection.

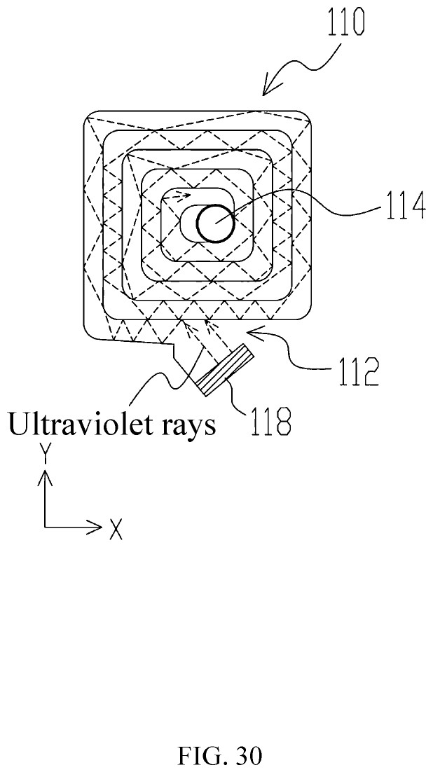

As shown in FIGS. 28 and 29, the vortex shape of the flow path 110 may be an algebraic spiral such as the Archimedean spiral, radiated spiral, hyperbolic spiral, or a lituus, or may be a logarithmic spiral (Bernoulli spiral or equiangular spiral). An outer shape of the flow path is not limited to a circular shape, and as shown in FIG. 30, the outer shape of the flow path may be substantially rectangle, may be a polygon such as triangle, pentagon, hexagon, etc. other than a rectangle, or may be an oval or ellipse.

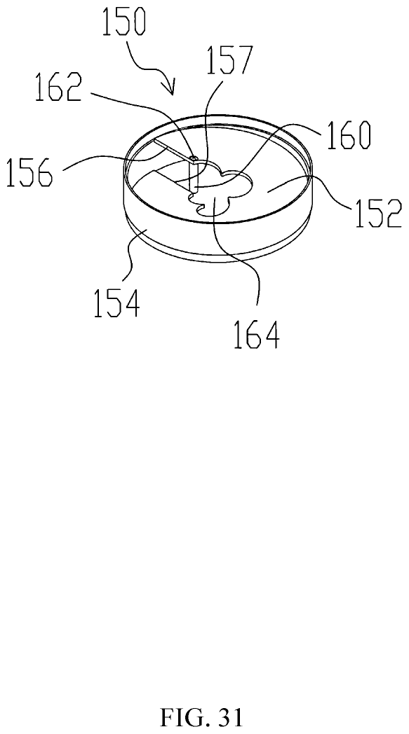

In the case of configuring a spiral flow path, a partial spiral flow path 150 shown in FIG. 31 may be applied. The partial spiral flow path 150 forms a spiral flow path corresponding to approximately a single turn and may include a spiral bottom surface 152, an axial position of which gradually changes according to a change in a circumferential position, an outer circumferential surface 154 for controlling a flow of the fluid in the radial direction in the partial spiral flow path 150, a support 160, a recess 162, an ultraviolet lamp insertion portion 164, and the like.

The spiral bottom surface 152 forms a spiral shape in which the axial position changes according to the circumferential direction. That is, as shown in FIG. 31, when the axial direction is directed to the vertical direction, the spiral bottom surface 152 has one end portion 156 which is position above the other end portion 157. The outer circumferential surface 154 forms a surface covering the circumferential direction of the partial spiral flow path 150.

The support 160 is a columnar member extending in parallel to the axial direction and has a length between the one end portion 156 and the other end portion 157. In other words, one end of the support 160 is fixed to a rear side of the spiral bottom surface 152 in the vicinity of the one end portion 156. The other end of the support 160 extends to substantially the same axial position as the other end portion 157. In addition, the support 160 may have a tubular shape with a through hole along the axial direction and may be configured to insert a wire or a harness thereinto.

The recess 162 is a groove on the spiral bottom surface 152, into which the support portion 160 may be inserted, and is formed on the spiral bottom surface 152 at a portion coinciding with the support 160 in the axial direction. That is, the recess 162 is positioned in the vicinity of the one end portion 156 on the spiral bottom surface 152.

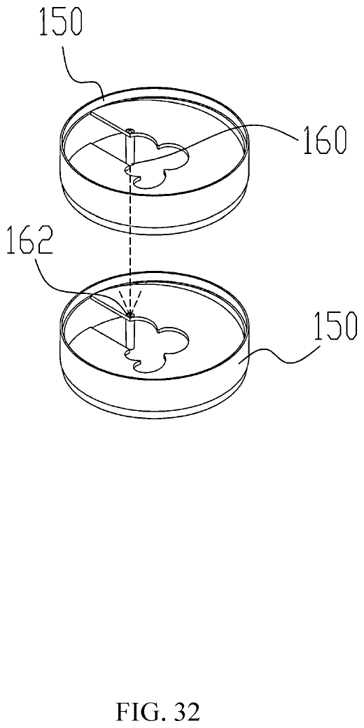

The support 160 and the recess 162 are disposed at the portion that is displaced from the center of the partial spiral flow path 150. Accordingly, when connecting two partial spiral flow paths 150, as shown in FIG. 32, the partial spiral flow paths 150 are aligned by matching a position of a tip end of the support 160 of one partial spiral flow path 150 with a position of the recess 162 of the other partial spiral flow path 150 along the axial direction.

Although one pair of the support 160 and the recess 162 is described, a plurality of pairs thereof may be provided.

The ultraviolet lamp insertion portion 164 is an opening formed in a central portion of the partial spiral flow path 150. In FIG. 31, the opening has an approximately trefoil shape with three circles aligned around the central portion of the partial spiral flow path 150 when seen in a direction parallel to the axial direction so that three ultraviolet light sources having a shape of a fluorescent lamp may be inserted. The opening formed by the ultraviolet lamp insertion portion 164 is not particularly limited, and may be appropriately set according to the shape of the ultraviolet light source, the number of those used, and the like. For example, the opening may have a circular shape, polygonal shape, Reuleaux polygonal shape, or a shape of a compound leaf (two leaves, four leaves, etc.) in which a plurality of circles is superimposed.

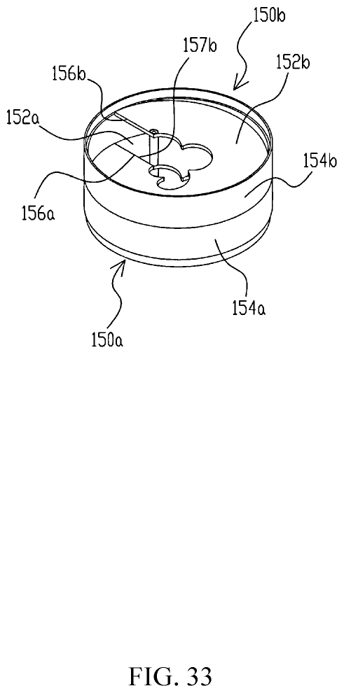

By connecting such plurality of partial spiral flow paths 150 along the axial direction, a spiral flow path having an arbitrary number of pitches may be formed. Herein, FIG. 33 is a perspective view illustrating two partial spiral flow paths 150a and 150b aligned in the axial direction, and the partial spiral flow path 150b is provided above the partial spiral flow path 150a in the axial direction.

As shown in FIG. 33, in a case of connecting the partial spiral flow paths 150a and 150b to each other, a turning direction of a spiral flow path of the partial spiral flow path 150b with respect to the partial spiral flow path 150a and mutual arrangement thereof are set so that one end portion 156a of a spiral bottom surface 152a of the partial spiral flow path 150a on one side and another end portion 157b of a spiral bottom surface 152b of the partial spiral flow path 150b on the other side are approximately continuously provided. This makes it possible to form a line of a spiral flow path with two turns.

As described above, by stacking the plurality of partial spiral flow paths 150, the line of the spiral may extend and the number of turns of the spiral may be increased by the number of partial spiral flow paths 150.

In addition, when a reflective surface is provided on the entire area of the spiral bottom surface 152 and an inner portion of the outer circumferential surface 154, the ultraviolet rays may be reflected multiple times within the spiral flow path formed by connecting the plurality of partial spiral flow paths 150. Therefore, the amount of emitted ultraviolet rays may be increased and the decreasing and eliminating efficiency of pathogenic microorganisms in the flow path may be improved, compared to a case where the ultraviolet rays are emitted to the inside of the flow path simply with the ultraviolet light source.

When the flow path is formed by a combination of the partial spiral flow paths 150, it is possible to improve ease of manufacture and also achieve mass production, compared to formation of the spiral flow path with one member. That is, when manufacturing the spiral flow path with one member, a manufacturing method such as using a three-dimensional (3D) printer, materials suitable for the method, and the like are limited due to a complicated shape of the flow path. In contrast, the shape of the partial spiral flow path 150 is simplified, and thus, the manufacturing method or material is not limited. Therefore, the flow path may be manufactured with a material having a high reflectivity with respect to ultraviolet rays at a low cost.

In addition, since the partial spiral flow path 150 has a simpler shape than a spiral flow path formed of one member, a reflective layer for reflecting the ultraviolet rays may be easily formed on the inner side of the partial spiral flow path 150. Also, a flow path with a spiral having a desired number of pitches may be configured by stacking a desired number of partial spiral flow paths in the axial direction, and thus, assemblability, workability, ease of manufacture, and the like are obtained. As a result, the spiral flow path with significantly excellent mass productivity may be provided by using the partial spiral flow path 150.

In addition, the spiral flow path is not limited to having a fixed diameter at any position in the axial direction. The spiral flow path may be set to have a shape with a diameter increasing or decreasing from one end portion to the other end portion in the axial direction, or may be set to have a shape in which one end portion and the other end portion in the axial direction have the same diameter and a middle portion has an increased or decreased diameter.

In addition, the ultraviolet leakage suppressor may be disposed to suppress leakage of the ultraviolet light from the suction portion and/or discharge portion. The ultraviolet leakage suppressor has a structure that does not disturb the flow of the fluid along the flow path while preventing the ultraviolet rays from being radiated to the outside of the device.

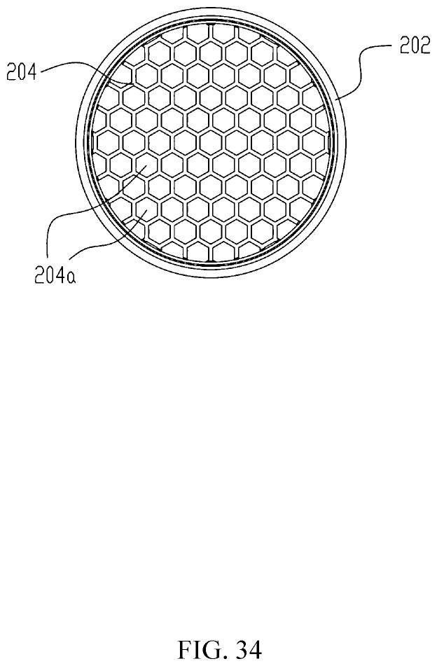

For example, an ultraviolet leakage suppressor 200 shown in FIG. 34 may be include an outer circumferential surface 202 connectable to the partial spiral flow path 150 and a suppressor 204 having a structure capable of suppressing the ultraviolet light from leaking to the outside of the device and transmitting the fluid on an inner side of the outer circumferential surface 202. The outer circumferential surface 202 has a cylindrical shape substantially connected to the outer circumferential surface of the partial spiral flow path 150. The suppressor 204 has a so-called honeycomb structure having a plurality of hexagonal holes 204a perforated in the axial direction.

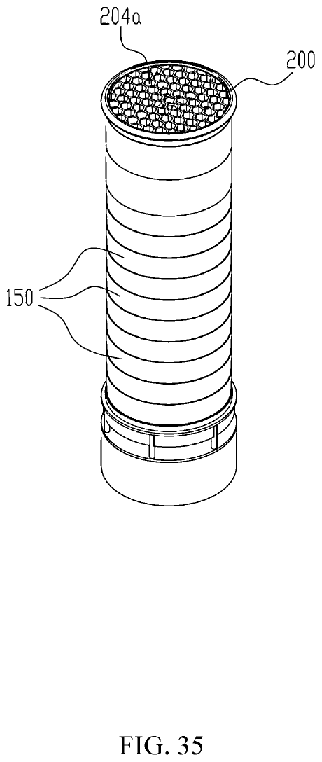

As shown in FIG. 35, when the ultraviolet leakage suppressor 200 is disposed on an uppermost portion of the flow path formed by combining the plurality of partial spiral flow paths 150, it is less likely to directly see the ultraviolet light source inserted into the ultraviolet lamp insertion portion 164, thereby reducing a risk of seeing the ultraviolet light by mistake. In addition, a shape of the hole 204a forming the honeycomb structure may be a polygonal shape other than a hexagonal shape or a porous shape.

As a fine hole of the suppressor 204 is provided or a depth of the hole thereof is increased, it is difficult to directly see the ultraviolet light source. That is, as the number of holes 204a is increased and the depth becomes deeper, the ultraviolet light easily reaches an inner wall in a direction other than the direction in which the hole 204a extends. Accordingly, an angle of the radiated ultraviolet rays capable of being transmitted through the suppressor 204 may be controlled and it is possible to make it impossible to visually recognize the ultraviolet light source in a direction other than directly looking into the hole 204a.

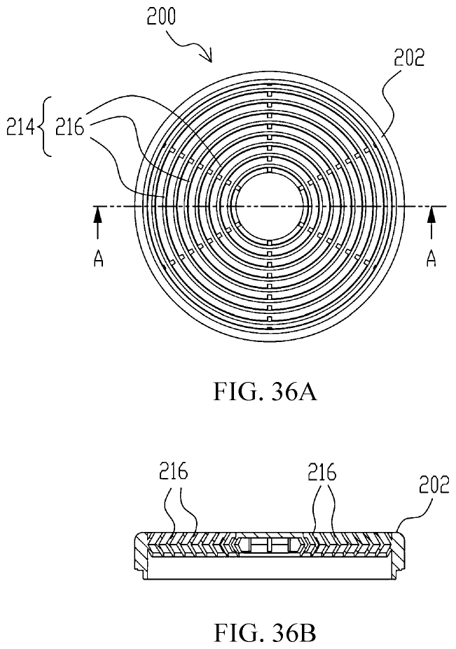

In addition, the ultraviolet leakage suppressor 200 may form a suppressing portion 214 by concentrically arranging a plurality of light shielding portions 216 forming an annular shape as shown in FIG. 36A. Herein, FIG. 36B is a cross-sectional view taken along line A-A of FIG. 36A. The light shielding portions 216 are set to have a cross section having a shape of V and to have an interval between adjacent light shielding portions 216 so that the ultraviolet light do not pass through between the light shielding portions 216. That is, in order to reliably shield the ultraviolet light, the interval, angle, and the like are set so that each light shielding portion 216 has a bent shape and the light shielding portions 216 are superimposed in a straight line view (a planar view) from the inner side toward the outer side.

The light shielding portion herein is described as being annular, but it is not limited thereto, and the light shielding portion may have a radial or linear shape.

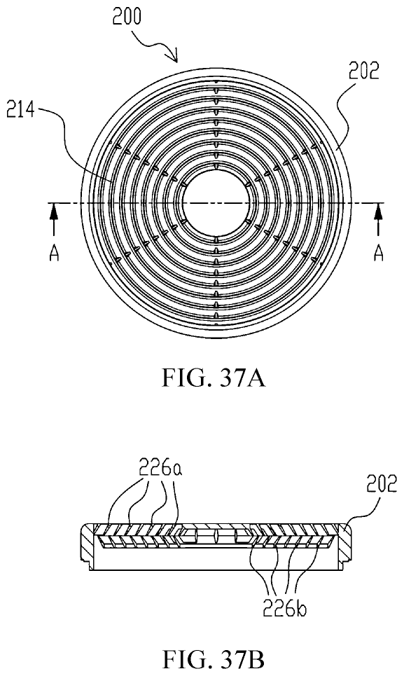

FIG. 37A is a plan view illustrating another example of the ultraviolet leakage suppressor and FIG. 37B is a cross-sectional view taken along a line A-A. The ultraviolet leakage suppressor 200 may include the suppressing portion 214 by arranging a plurality of two types of inclined surfaces 226a and 226b forming an annular shape, respectively. That is, the suppressing portion 214 may be configured by disposing first inclined surfaces 226a on an upper side (one side) in the axial direction and disposing second inclined surfaces 226b on a lower side in the axial direction (the other side).

Each of the first inclined surface 226a and the second inclined surface 226b has a cross section inclined with respect to the axial direction, and the first inclined surface 226a and the second inclined surface 226b have different inclinations.

Specifically, the first inclined surface 226a is inclined so that one side in the axial direction is inclined toward an axial center, and the second inclined surface 226b is inclined so that the other side in the axial direction is inclined toward the axial center. In addition, the first inclined surface 226a and the second inclined surface 226b are spaced apart from each other, and the arrangement position is shifted in the radial direction so that the first inclined surface 226a exists on an extension line of an inclination direction of the second inclined surface 226b. That is, as shown in FIG. 37B, the first inclined surface 226a and the second inclined surface 226b are disposed to be alternately aligned along the radial direction.

In addition, an interval between the first inclined surface 226a and the second inclined surface 226b, an angle thereof, or the like are set in order to reliably shield the ultraviolet light radiated between the first inclined surfaces 226a (or between the second inclined surfaces 226b) by the second inclined surfaces 226b (or the first inclined surfaces 226a).

Even by providing the ultraviolet leakage suppressor 200, the ultraviolet light may be shielded reliably without disturbing the flow of the fluid. As a result, it is possible to make it impossible to see the ultraviolet light source directly from the outside of the toxic subject decreasing and eliminating device.

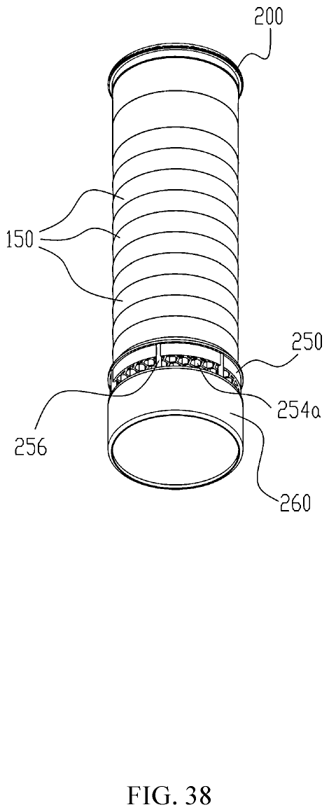

In addition, each ultraviolet leakage suppressor described above may be disposed not only on the uppermost portion of the flow path, but also on a lowermost portion of the flow path. That is, as shown in FIG. 38, the ultraviolet leakage suppressor 250 may be disposed on the lowermost portion of the flow path or on an upper portion of a base 260 of the device. The ultraviolet leakage suppressor 250 in this case includes a plurality of holes 254a forming a honeycomb structure and transmitting a fluid, and a spacer 256 for providing a gap between the holes 254a and the base 260.

The ultraviolet leakage suppressor 250 may be positioned on an upper portion of the device and the discharge or suction of the fluid may be performed through the gap provided by the spacer 256. In addition, the ultraviolet leakage suppressor 250 may have a surface opposite to the hole 254a with the spacer 256 interposed therebetween as a reflective surface and may reflect the ultraviolet light passed through the hole 254a by the reflective surface and introduce the ultraviolet light to the flow path side again. A shape of the reflective surface is not particularly limited, and may be planar, spherical, or curved. Particularly, when the reflective surface is set to have a concave and spherical shape, a risk of seeing the ultraviolet light may be reduced because the ultraviolet light is not reflected to the outside of the device.

### REFERENCE SIGNS LIST

1, 20, 50: Toxic subject decreasing and eliminating device
2, 34, 112: Suction portion
4, 36, 114: Discharge portion
6: Flow path
8, 26, 80, 118: Ultraviolet light source
10: Flow generator
12, 41, 68: Guide plate
16, 22: Housing
18: Concave reflector
24, 64: Flow path portion
28, 70: Blower
30, 56: Ceiling
32: Bottom portion
40, 66: Air flow path
42: Installation space
44: Partition plate
50: Eliminating device
51: Housing
52: Blade
54: Driving transmitter
55: Rotating body
72: Rotating shaft
74: Plate-shaped blades
82: Inner annular surface
84: Outer annular surface
90: Motor
100: Cyclone chamber
102: Dust collecting portion
110: Vortex-shaped flow path
116: Fluid guide surface
150: Partial spiral flow path
152: Spiral bottom surface
154: Outer circumferential surface
156: One end portion
157: Other end portion
160: Support

1. A toxic subject decreasing and eliminating device comprising:
a flow path that communicates a suction portion configured to suck a fluid and a discharge portion configured to discharge the fluid with each other;
a decreasing and eliminating means including an ultraviolet light source for decomposing and/or inactivating and/or sterilizing a subject contained in the fluid flowing down in the flow path; and
a reflective surface that reflects ultraviolet ray from the decreasing and eliminating means toward the flow path, wherein the reflective surface reflects, with high order, an ultraviolet ray emitted from the ultraviolet light source toward the flow path at multiple times, to highly amplify a density of ultraviolet rays in the flow path.

2. The device of claim 1, further comprising:
a flow generating means for generating a flow of the fluid along the flow path from a side of the suction portion toward a side of the discharge portion.

3. The device of claim 2, wherein the flow generating means has at least one or more fan structures selected from an axial-flow fan, a centrifugal fan, a mixed flow fan, a centrifugal axial-flow fan, a vortex-flow fan, or a tangential fan.

4. The device of claim 3, wherein the flow generating means is disposed in a vicinity of the suction portion and/or the discharge portion.

5. The device of claim 2,
wherein the flow generating means has one or more fan structures, and
wherein the one or more fan structures are fixed to a single rotating shaft.

6. The device of claim 2, wherein the flow generating means comprises one or more fan structures, a rotating shaft, and a driving motor.

7. The device of claim 2, further comprising:
at least one sensor of a temperature sensor, a humidity sensor, a human sensor, or a pollution sensor,
wherein the flow generated by the flow generating means is controlled based on detection by the at least one sensor.

8. The device of claim 1, wherein the flow path reciprocates in a predetermined direction.

9. The device of claim 8, wherein the predetermined direction is a horizontal direction and/or a vertical direction.

10. The device of claim 8, wherein the predetermined direction is a direction parallel to ultraviolet rays emitted by the decreasing and eliminating means.

11. The device of claim 1, wherein the flow path comprises a portion extending in a curved shape and/or meander shape.

12. The device of claim 11, wherein the portion extending in the curved shape forms a spiral shape or a vortex shape.

13. The device of claim 1, wherein the flow path has a distance that is an integer multiple or more of a straight line distance between the suction portion and the discharge portion.

14. The device of claim 1, wherein the flow path is configured with a flow path defining means.

15. The device of claim 14, wherein the flow path defining means is configured to comprise any one or more of a portion of the decreasing and eliminating means, a portion of an inner surface of a housing, and a guide plate.

16. The device of claim 1, wherein the ultraviolet light source comprises an ultraviolet lamp or an ultraviolet light-emitting diode (LED).

17. The device of claim 16, wherein the ultraviolet lamp is formed as a cylindrical tube.

18. The device of claim 17,
wherein a reflective surface has a concave cross section having an elliptical arc shape, and
wherein the ultraviolet lamp is disposed at a focal point position of an ellipse that forms the elliptical arc shape of the reflective surface.

19. The device of claim 16, wherein a plurality of ultraviolet LEDs is arranged approximately in a straight line or arranged to be aligned vertically and/or horizontally in a plane.

20. The device of claim 1, wherein the decreasing and eliminating means is integrally configured with the flow path.

21. The device of claim 1, wherein the discharge portion is configured to discharge the fluid toward a suction region by the suction portion.

22. The device of claim 1, wherein the discharge portion is configured to discharge the fluid toward a region different from a suction region by the suction portion.

23. The device of claim 1, wherein a flow velocity of a fluid discharged by the discharge portion is slower than a flow velocity of a fluid sucked by the suction portion.

24. The device of claim 1, wherein an area of a discharge port of the fluid in the discharge portion is larger than an area of a suction port of the fluid in the suction portion.

25. The device of claim 1, further comprising:
a housing having an approximately tubular shape and elongated shape, and
wherein one of the suction portion and the discharge portion is disposed on one end side with respect to a longitudinal central portion of the housing, and the other one is disposed on an other end portion with respect to the longitudinal central portion of the housing.

26. The device of claim 1,
wherein the suction portion performs high-speed suction, and
wherein the discharge portion performs low-speed discharge.

27. The device of claim 1,
wherein the suction portion performs low-speed suction, and
wherein the discharge portion performs high-speed discharge.

28. The device of claim 1, wherein the suction portion comprises a suction port for sucking the fluid from a wide area.

29. The device of claim 1, wherein the suction portion comprises a suction port for sucking the fluid from a single direction.

30. The device of claim 1, wherein the suction portion allows the sucked fluid to flow down to the flow path as a jet flow.

31. The device of claim 1, wherein the discharge portion comprises a discharge port for discharging the fluid to a wide area.

32. The device of claim 1, wherein the discharge portion comprises a discharge port for discharging the fluid in a single direction.

33. The device of claim 1,
wherein the discharge portion comprises a continuous or intermittent exhaust port extending in one direction, and
wherein an air curtain is generated by exhaust from the exhaust port.

34. The device of claim 1, wherein the discharge portion is configured to discharge a jet flow.

35. The device of claim 1, wherein the flow path is formed of an ultraviolet transmitting material.

36. The device of claim 1,
wherein the reflective surface is disposed on a portion facing the ultraviolet light source with the flow path interposed therebetween.

37. The device of claim 1, further comprising:
a second decreasing and eliminating means for decreasing and eliminating a subject,
wherein the second decreasing and eliminating means comprises an electric field generating means for generating an electric field in a flow path, a heating means for heating inside of the flow path, and/or an ion generating means for generating ions.

38. The device of claim 1, wherein a filter for collecting foreign matters contained in the fluid is provided in the flow path.

39. The device of claim 1, wherein the flow path comprises a cyclone portion for separating foreign matters contained in the fluid from the flow path.

40. The device of claim 1, further comprising:
a partition configured to partition a space around the device.

41. The device of claim 1, wherein the toxic subject decreasing and eliminating device is embedded in separate equipment.

42. The device of claim 41, wherein the separate equipment is a roof, seat backrest, seat headrest, concrete panel, air conditioner, table, desk, chair, elevator, plant, septic tank, or pipe.

43. The device of claim 1,
wherein a plurality of guide plates for partitioning the flow path into a plurality of regions concentrically is disposed in the flow path at intervals in a flow direction of the fluid,
wherein each guide plate has a communication path on one end portion or an other end portion along a reciprocating direction of the fluid, and
wherein the fluid in the flow path is allowed to flow down from the suction portion toward the discharge portion while flowing in the reciprocating direction and flowing to an inner side in a radial direction through the communication path.

44. The device of claim 43, wherein each region of the plurality of regions partitioned by the plurality of guide plates has the same cross-sectional area.

45. The device of claim 43, wherein each region of the plurality of regions partitioned by the plurality of guide plates has a cross-sectional area set to be narrower toward the inner side in the radial direction.

46. The device of claim 43, wherein each region of the plurality of regions partitioned by the plurality of guide plates has a cross-sectional area set to be wider toward the inner side in the radial direction.

47. The device of claim 1,
wherein the suction portion and the discharge portion are disposed in plurality intermittently along a circumferential direction,
wherein the flow path is partitioned into a plurality of sections in the circumferential direction and communicates the suction portion and the discharge portion directed in the same direction with each other, and
wherein the fluid sucked from one side by the suction portion is discharged toward the one side through the discharge portion.

48. The device of claim 47, wherein the decreasing and eliminating means is disposed on a portion surrounded by the flow path.

49. The device of claim 1, wherein the fluid is a gas, liquid, and/or powder.

50. The device of claim 1, wherein the subject is bacteria, viruses and/or harmful molecules.

51. A toxic subject decreasing and eliminating device comprising:
a flow path that communicates a suction portion configured to suck a fluid and a discharge portion configured to discharge the fluid with each other, and defines a path in a spiral shape or vortex shape;
a reflective layer that is disposed in the flow path and extends along a direction in which the fluid flows; and
a decreasing and eliminating means including an ultraviolet light source that is disposed on the suction portion and/or the discharge portion and decomposes and/or inactivates and/or sterilizes a subject contained in the fluid flowing down in the flow path with ultraviolet rays,
wherein ultraviolet rays emitted from the decreasing and eliminating means are reflected with high order at multiple time in the flow path by the reflective layer and a density of ultraviolet rays in the flow path is highly amplified.

52. The device of claim 51, wherein the reflective layer is continuously or intermittently formed in the flow path.

53. The device of claim 12, wherein the flow path having the spiral shape is formed by stacking partial spiral flow paths each having the spiral shape for one turn in an axial direction.

54. The device of claim 53, wherein the partial spiral flow paths comprise:
an insertion portion that is formed in a central portion and through which the decreasing and eliminating means is inserted;
a support that fits into another communicating partial spiral flow path and extends in the axial direction; and
a recess into which the support provided in the another communicating partial spiral flow path fits.

55. A toxic subject decreasing and eliminating device comprising:
a flow path that communicates a suction portion configured to suck a fluid and a discharge portion configured to discharge the fluid with each other;
a reflective layer that is disposed in the flow path and extends along a direction in which the fluid flows; and
a decreasing and eliminating means including an ultraviolet light source that is disposed on the suction portion and/or the discharge portion and decomposes and/or inactivates and/or sterilizes a subject contained in the fluid flowing down in the flow path with ultraviolet rays,
wherein ultraviolet rays emitted from the decreasing and eliminating means are reflected with high order at multiple time in the flow path by the reflective layer and a density of ultraviolet rays in the flow path is highly amplified.

56. The device of claim 1, wherein an ultraviolet leakage suppressor for suppressing ultraviolet light leaking to outside of the device and transmitting the fluid is disposed on a side of the suction portion and/or a side of the discharge portion.

57. The device of claim 56, wherein the ultraviolet leakage suppressor comprises a plurality of holes forming a honeycomb structure.

58. The device of claim 56, wherein the ultraviolet leakage suppressor comprises a light shielding surface having a bent cross section.

59. The device of claim 56,
wherein the ultraviolet leakage suppressor comprises a first inclined surface and a second inclined surface, and
wherein the first inclined surface and the second inclined surface are disposed at different inclination angles.

60. The device of claim 56,
wherein the ultraviolet leakage suppressor comprises a first inclined surface and a second inclined surface, and
wherein the first inclined surface and the second inclined surface are spaced apart from each other and provided to be shifted so that one inclined surface exists on an extension line of the other inclined surface in an inclination direction.
